(19) United States
(12) Patent Application Publication
(10) Pub. No.: US 2026/0242424 A1
(43) Pub. Date: Aug. 20, 2026

(54) A METHOD OF ENHANCED SEPARATION OF FULL ADENO-ASSOCIATED VIRUS (AAV) CAPSIDS

(71) Applicant: Sartorius BIA Separations d.o.o., Ajdovscina (SI)

(72) Inventors: Aleš ŠTRANCAR, Ajdovscina (SI); Timotej ŽVANUT, Ajdovscina (SI); Rok ZIGON, Ajdovscina (SI); Maja LESKOVEC, Ajdovscina (SI); Andreja Gramc LIVK, Ajdovscina (SI); Janja Merkelj KOREN, Ajdovscina (SI)

(21) Appl. No.: 19/489,524

(22) PCT Filed: Jun. 10, 2024
(86) PCT No.: PCT/EP2024/065910

§ 371 Date: Dec. 2, 2025

(30) Foreign Application Priority Data
Jun. 9, 2023 (EP) 23178503.1

(51) Int. Cl. C07K 14/005; B01D 15/36; B01D 15/38; C07K 1/18

(52) U.S. Cl. CPC C07K 14/005; B01D 15/363; B01D 15/3847; C07K 1/18; C12N 2750/14151

(57) ABSTRACT

A method for enriching full Adeno-Associated Virus (AAV) capsids from a mixture comprising full Adeno-Associated Virus (AAV) capsids, partially filled and/or empty AAV capsids by means of chromatography, comprising the steps of
contacting the mixture with a strong or weak anion exchanger material
eluting the loaded mixture by means of a neutral to an alkaline buffer comprising an organic modifier having a relative measure of solvent polarity from 0.4 to 0.8,
and collecting a fraction enriched with full AAV capsids.

The invention is related to a method for enriching full Adeno-Associated Virus (AAV) capsids from a mixture comprising full AAV capsids, partially filled and/or empty AAV capsids by means of chromatography The method of the invention is particularly useful in yielding enriched full AAV capsid fractions with depleted contaminants such as empty, partially filled, heavy, damaged AAV capsids, capsids aggregates, etc.

### BACKGROUND OF THE INVENTION

Gene therapy is a promising medical field which focuses on the genetic modification of cells to produce a therapeutic effect or the treatment of disease by repairing or reconstructing defective genetic material. The genetic material is administered to the patient suffering from a disorder caused by defective genes. One method of administration of the curative genetic material is performed by using vectors for capsids of viruses or virus-like particles, in particular adeno-associated virus (AAV) capsids.

AAVs are widely used vectors in gene therapy, primarily due to its safety profile and efficient transduction to various target tissues. Production of AAV viral vectors is a complex process and requires innovative approaches to meet stringent safety and efficacy requirements, and strict clinical and market demands.

Despite extensive clinical applications improved purification approaches are required to remove product and process related impurities and assuring a high AAV product quality and potency throughout the production process.

Both, empty capsids and capsids partially filled with intended vector genome both represent product related impurities and are technically challenging to separate from the target vector during downstream purification. Removal of empty capsids from the preparations of AAV and to maximization of the ratio between full and empty capsids is one of the goals of purification, which addresses safety and regulatory recommendations for AAV-based gene therapy [1-3].

Dependent on the exact packed sequence, partially filled capsids, may still contribute to target cell transduction [4]. However, AAV capsids containing host cell and/or helper DNA and product related impurities may represent an immunological risk to patients [5].

This has led to development and evaluation of many different materials and methods purify AAVs. For example, metal affinity chromatography has been used to purify AAV capsids [6]. However, it does not discriminate between empty and full capsids and is serotype dependent. A crude sample containing the desired AAV capsids and contaminants is contacted with the metal affinity material. In principle, AAV capsids bind to the affinity material whereas contaminants do not. The unbound contaminants are washed away and the AAV capsids are recovered by chemically disrupting the interaction between the affinity material and the AAV.

Downstream processing, including purification, remains one of the main bottlenecks in adenoviral associated vector manufacturing. BIA Separations, a Sartorius company offers a platform for purification of adenoviral associated vaccines using market leading monolithic chromatographic columns and an analytical toolbox for process monitoring of adenoviral associated vaccine production.

Simplified purification of AAV capsids consists of typical downstream steps, including combined lysis, clarification, tangential flow filtration (TFF), and chromatographic capture on pre-packed monolithic sulfonate (SO₃) column and enrichment of full AAV capsids by pre-packed monolithic quaternary amine (QA) column.

Although this process provides adeno-associated virus of pharmaceutical grade, it is desirable to further improve purity of AAV for gene therapy, in particular to separate empty as well as only partially filled AAV capsids and/or carrying impurities like DNA or other contaminants from therapeutically needed full AAV capsids loaded with genetic material, e. g. plasmids, and freed from contaminants.

### OBJECT OF THE INVENTION

One object of the invention is to provide a method for separating full Adeno-Associated Virus (AAV) capsids from empty and partially filled AAV capsids.

A further object of the invention is to provide a method for obtaining an enriched fraction of full AAV capsids from a mixture comprising full AAV capsids and empty, partially filled, heavy, damaged AAV capsids, capsid aggregates, etc.

Another object is to provide a method yielding full AAV capsids as free as possible of contaminants, such as DNA, more specifically hcDNA and pDNA.

Still another object is to provide a method which can be used analytically as well in a preparative scale.

### SUMMARY OF THE INVENTION

Subject matter of the present invention is a method for enriching full Adeno-Associated Virus (AAV) capsids from a mixture comprising full Adeno-Associated Virus (AAV) capsids, partially filled and/or empty AAV capsids by means of chromatography, comprising the steps of
contacting the mixture with a strong or weak anion exchanger material
eluting the loaded mixture by means of a neutral to an alkaline buffer comprising an organic modifier having a relative measure of solvent polarity from 0.4 to 0.8,
and collecting a fraction enriched with full AAV capsids.

The relative measure of solvent polarity, RPM is defined by equation:

$$R P M = {E}_{T} ( n - hexane ) - {E}_{T} / {E}_{T} ( n - hexane ) as described by Dukiie at al . \qquad [ 8 ]$$

E_{T}—transition energies [kJ/mol]

RPM values in Table 1 are negative, however for correlation analyses it is sufficient to use absolute RPM values [7].

The method of the invention can advantageously be used both for analytical purposes and for preparative manufacturing of full AAV capsids.

According to one embodiment of the invention the organic modifier can be selected from the group consisting of methanol, 1,3-propanediol, 1,2-propanediol, N-methylformamide, diethylene glycol, triethylene glycol, 1,3-butanediol, 2-propyn-1-ol (propargyl alcohol), 2-methoxyethanol, 2-propen-1-ol (allyl alcohol), N-methylacetamide, ethanol, 2-aminoethanol, acetic acid, benzyl alcohol, 1-propanol, 1-butanol, 2-hydroxymethylfuran (furfuryl alcohol), 2-phenylethanol, 1-pentanol, 2-methyl-1-propanol (isobutyl alcohol), 1-hexanol, 2-propanol, 3-phenyl-1-propanol, 1-heptanol, 1-octanol, cyclopentanol, 1-decanol, 2,6-dimethylphenol (2,6-xylenol), 2-butanol, 3-methyl-1-butanol (isoamyl alcohol), cyclohexanol, 1-dodecanol, 1-phenylethanol, acrylonitrile, 4-methyl-1,3-dioxolan-2-one (propylene carbonate), 2-pentanol, nitromethane, acetonitrile, dimethyl sulfoxide, methyl acrylate, aniline, tetra-N-hexylammonium benzoate, tetrahydrothiophene 1,1-dioxide (sulfolane), 2-methyl-2-propanol (tert-butyl alcohol), acetic anhydride, N,N-dimethylformamide, N,N-dimethylacetamide, propionitrile and nitroethane.

Preferred organic modifiers are acetonitrile, propylene carbonate, 2-propanol, 1-butanol, 1-propanol, t-butanol, ethanol, methanol, and mixtures thereof. An embodiment of the invention is a method for enriching full Adeno-Associated Virus (AAV) capsids from a mixture comprising full Adeno-Associated Virus (AAV) capsids, partially filled and/or empty AAV capsids by means of chromatography, comprising the steps of
contacting the mixture with a strong or weak anion exchanger material
eluting the loaded mixture by means of a neutral to an alkaline buffer comprising an organic modifier selected from the group consisting of methanol, 1,3-propanediol, 1,2-propanediol, N-methylformamide, diethylene glycol, triethylene glycol, 1,3-butanediol, 2-propyn-1-ol (propargyl alcohol), 2-methoxyethanol, 2-propen-1-ol (allyl alcohol), N-methylacetamide, ethanol, 2-aminoethanol, acetic acid, benzyl alcohol, 1-propanol, 1-butanol, 2-hydroxymethylfuran (furfuryl alcohol), 2-phenylethanol, 1-pentanol, 2-methyl-1-propanol (isobutyl alcohol), 1-hexanol, 2-propanol, 3-phenyl-1-propanol, 1-heptanol, 1-octanol, cyclopentanol, 1-decanol, 2,6-dimethylphenol (2,6-xylenol), 2-butanol, 3-methyl-1-butanol (isoamyl alcohol), cyclohexanol, 1-dodecanol, 1-phenylethanol, acrylonitrile, 4-methyl-1,3-dioxolan-2-one (propylene carbonate), 2-pentanol, nitromethane, acetonitrile, dimethyl sulfoxide, methyl acrylate, aniline, tetra-N-hexylammonium benzoate, tetrahydrothiophene 1,1-dioxide (sulfolane), 2-methyl-2-propanol (tert-butyl alcohol), acetic anhydride, N,N-dimethylformamide, N,N-dimethylacetamide, propionitrile and nitroethane,
and collecting a fraction enriched with full AAV capsids.

In another embodiment of the invention the buffer may comprise alkaline earth metal salts in particular salts of magnesium or calcium and/or mixtures thereof.

In still another embodiment of the invention the alkaline earth metal salts can be magnesium acetate or formate, or calcium acetate or formate or mixtures thereof and/or their more kosmotropic alternatives. Preferred more kosmotropic alternatives are magnesium and calcium salts of inorganic acids, organic acids or organic hydroxy acids amino acids or polycarboxylic acids having up to 10 carbon atoms, for example oxalate or citrate.

In yet another embodiment of the invention the buffer may have a pH value of from about pH 7.0 to about pH 10.5, in particular of from about pH 7.5 to about pH 9.50.

In a further embodiment of the invention the buffer can comprise isotonic substances selected from the group consisting of sucrose, sorbitol, mannitol, and xylitol.

According to another embodiment of the invention the strong or weak anion exchanger material can be a strong or weak anion exchanger material with hydrogen bond properties and compounded with positively charged metal affinity ligand as a multimodal material, a monolith anion exchanger or multimodal material, a particulate anion exchanger or multimodal material, and/or an anion exchanger or multimodal material arranged in membranes, and/or particle packed anion exchanger or multimodal columns and/or fibre chromatography anion exchanger or multimodal fibre columns.

Multimodal chromatography also known as mixed-mode chromatography (MMC), refers to chromatographic methods that utilize more than one form of interaction between the stationary phase and analytes in order to achieve their separation [16,17,18]. MMC can be classified into physical MMC and chemical MMC. In the former method, the stationary phase is constructed of two or more types of packing materials. In the chemical method, one type of packing material containing two or more functionalities is used. An approach is to connect two commercial columns in series, which is termed a “tandem column”. Another approach is “biphasic column”, by packing two stationary phases separately in two ends of the same column. A further approach is to homogenize two or more different types of stationary phases in a single column, which is termed a “hybrid column” or “mixed-bed column”.

According to still another embodiment of the invention the AAV may be selected from serotypes selected from the group of AAV1, AAV2, AAV3, AAV4, AAV5, AAV6, AAV7, AAV8, AAV9, AAVrh.10, AAV11, AAV12 and of different serotypes such as hybrid serotypes. The AAV serotype analysed by the method of invention may be a recombinant hybrid serotype like AAV2/8 or another hybrid serotype, chimeras, surface modified AAVs and any synthetic derived AAV like particles.

A chimera or chimeric virus is a virus that contains genetic material derived from two or more distinct viruses.

Surface modified virus are known and for example described in [20].

Synthetic derived AAV like particles are known and for example described in [21].

Subject matter of the invention is also an aqueous solution having a neutral to alkaline pH comprising buffer substances and an organic modifier having a relative polarity measure (RPM) of solvent equivalent from 0.4 to 0.8.

According to another embodiment of the aqueous solution of the invention the organic modifier can be selected from the group consisting of methanol, 1,3-propanediol, 1,2-propanediol, N-methylformamide, diethylene glycol, triethylene glycol, 1,3-butanediol, 2-propyn-1-ol (propargyl alcohol), 2-methoxyethanol, 2-propen-1-ol (allyl alcohol), N-methylacetamide, ethanol, 2-aminoethanol, acetic acid, benzyl alcohol, 1-propanol, 1-butanol, 2-hydroxymethylfuran (furfuryl alcohol), 2-phenylethanol, 1-pentanol, 2-methyl-1-propanol (isobutyl alcohol), 1-hexanol, 2-propanol, 3-phenyl-1-propanol, 1-heptanol, 1-octanol, cyclopentanol, 1-decanol, 2,6-dimethylphenol (2,6-xylenol), 2-butanol, 3-methyl-1-butanol (isoamyl alcohol), cyclohexanol, 1-dodecanol, 1-phenylethanol, acrylonitrile, 4-methyl-1,3-dioxolan-2-one (propylene carbonate), 2-pentanol, nitromethane, acetonitrile, dimethyl sulfoxide, methyl acrylate, aniline, tetra-N-hexylammonium benzoate, tetrahydrothiophene 1,1-dioxide (sulfolane), 2-methyl-2-propanol (tert-butyl alcohol), acetic anhydride, N,N-dimethylformamide, N,N-dimethylacetamide, propionitrile and nitroethane.

Preferred organic modifiers are acetonitrile, 1-butanol, t-butanol, propylene carbonate, isopropanol, ethanol, methanol and propanol.

According to yet another embodiment of the aqueous solution of the invention the solution may comprise alkaline earth metal salts.

According to still another embodiment of the aqueous solution of the invention the alkaline earth metal salts can be salts of magnesium or calcium and mixtures thereof, in particular magnesium acetate or formate and/or calcium acetate or formate and/or their more kosmotropic alternatives. Preferred more kosmotropic alternatives are magnesium and calcium salts of inorganic acids, organic acids or organic hydroxy acids or amino acids or polycarboxylic acids having up to 10 carbon atoms, for example oxalate or citrate.

According to a further embodiment of the aqueous solution of the invention the buffer substances buffering an aqueous solution in a range from pH 6 to pH 12 in particular buffer substances may be selected from the group consisting of 2-[bis(2-hydroxyethyl)amino]-2-(hydroxymethyl)propane-1,3-diol (Bis-Tris), 2,2′,2″-Nitrilotriacetic acid (ADA), 2-[(2-Amino-2-oxoethyl)amino]ethane-1-sulfonic acid (ACES), 2,2′-(Piperazine-1,4-diyl)di(ethane-1-sulfonic acid) (PIPES), 2-Hydroxy-3-(morpholin-4-yl)propane-1-sulfonic acid (MOPSO), 2,2′-[Propane-1,3-diylbis(azanediyl)]bis[2-(hydroxymethyl)propane-1,3-diol](BTP), N,N-Bis(2-hydroxyethyl)-2-aminoethanesulfonic acid (BES), 3-(Morpholin-4-yl)propane-1-sulfonic acid (MOPS), 2-{[1,3-Dihydroxy-2-(hydroxymethyl)propan-2-yl]amino}ethane-1-sulfonic acid (TES), 2-[4-(2-Hydroxyethyl)piperazin-1-yl]ethane-1-sulfonic acid (HEPES), 3-[N,N-Bis(2-Hydroxyethylamino)-2-Hydroxy-1-Propanesulfonic Acid (DIPSO), 4-(4-Morpholinyl)butanesulfonic acid (MOBS), 2-Hydroxy-3-[tris(hydroxymethyl)methylamino]-1-propanesulfonic acid (TAPSO), 2-Amino-2-(hydroxymethyl)-1,3-propandiol (Trizma), 4-(2-hydroxyethyl)piperazine-1-(2-hydroxypropane-3-sulfonic acid) (HEPPSO), Piperazine-N,N′-bis(2-hydroxypropanesulfonic acid), (POPSO), Triethylamine (TEA), 4-(2-Hydroxyethyl)-1-piperazine-propanesulfonic acid (EPPS), N-tris(hydroxymethyl)methylglycine (Tricine), N,N-Bis(2-hydroxyethyl)glycine (Bicine), N-(2-Hydroxyethyl)piperazine-N′-(4-butanesulfonic acid) (HEPBS), N-tris(Hydroxymethyl)methyl-4-aminobutanesulfonic acid (TAPS), 2-Amino-2-methyl-1,3-propanediol (AMPD), N-tris(Hydroxymethyl)methyl-4-aminobutanesulfonic acid (TABS), N-(1,1-Dimethyl-2-hydroxyethyl)-3-amino-2-hydroxypropanesulfonic acid (AMPSO), 2-(N-Cyclohexylamino)ethanesulfonic acid (CHES), 3-Cyclohexylamino-2-hydroxypropanesulfonic acid sodium salt (CAPSO), N-(1,1-Dimethyl-2-hydroxyethyl)-3-amino-2-hydroxypropanesulfonic acid (AMPS), 3-(Cyclohexylamino)-1-propanesulfonic acid (CAPS) and 4-[Cyclohexylamino]-1-butanesulfonic acid (CABS).

According to another embodiment of the aqueous solution of the invention the buffer can comprise isotonic additives in particular isotonic additives selected from the group consisting of sucrose, sorbitol, mannitol, and xylitol.

The buffer can also comprise non-ionic surfactants which are useful to suppress unwanted effects e. g. interactions between components of the mixture comprising AAV capsids. In particular so called poloxamers, such as poloxamer 188 can be used.

Subject matter of the invention is also the use of the aqueous solution of the invention for separating full AAV capsids from empty AAV capsids according to the method the invention.

Table 1 depicts some organic solvents and their respective RPM values. The Table is derived from reference [7].

### BRIEF DESCRIPTION OF THE FIGURES

FIG. 1 depicts the resolution of the separation of full/empty AAV capsids of different organic modifiers.

FIGS. 2A and 2B depict the influence of organic modifier on the resolution of full/empty AAV capsids. FIG. 2C depicts the influence of organic modifier the presence of magnesium chloride (MgCl₂), acetate (MgAc₂), and formate (MgFor₂) on the resolution of full/empty AAV capsids.

FIG. 3 depicts the influence of a preferred organic modifier on the resolution of the separation of full/empty AAV capsids compared to poloxamer 188.

FIG. 4 depicts the influence of percentage of organic modifier on the separation of empty/full AAV capsids.

FIG. 5 depicts the influence of the concentration of Mg²⁺ ions regarding resolution in the elution buffer regarding resolution.

FIG. 6 depicts the pH range of elution buffer used for separation of empty/full AAV8 capsids.

FIG. 7 depicts the influence of pH on the separation of AAV8 and AAV9 serotype capsids.

FIG. 8A depicts a preparative (purification) run of a sample containing different populations of AAV capsids. For the collected fractions an analytical run was performed showing multiple detector results, FIGS. 8B, 8C, 8D and 8E.

FIG. 9 depicts the influence of a preferred organic modifier on the resolution of the separation of full/empty AAV capsids on a weak anion exchanger monolith.

FIG. 10 depicts the influence of a preferred organic modifier on the resolution of the separation of full/empty AAV capsids on a multimodal exchanger monolith or membrane.

FIGS. 11A and 11B depict the influence of higher percentage of organic modifier on AAV capsids separation, particularly on the baseline separation of empty and partially capsids from full capsids.

FIG. 12 depicts elution fractions from preparative run, analysed by orthogonal density gradient ultracentrifugation coupled with PATfix™ (Sartorius BIA Separations) multiple detector setup.

FIG. 13 depicts the comparison of AAV capsids separation using QA column and anion exchanger membrane adsorber.

FIG. 14 depicts the resolution of the separation of full/empty AAV capsids of different loading and elution strategies.

FIG. 15 depicts a chromatogram showing the tryptophan fluorescence and light scattering resolution of the separation of harvest AAV8 and full/empty AAV8 capsids from the same batch by using a two-dimensional chromatographic system.

### DETAILED DESCRIPTION OF THE INVENTION

The term “resolution” is known to the person skilled in the art. Resolution is calculated by dividing the difference in peak retention times between different chromatographic peaks by the peak width at a half height of the respective peaks using the formula below [9].

$$R = 1.18 ( {t}_{R 2} - {tr}_{R 1} ) / ( {W}_{h 1} + {W}_{h 2} ) , whereby {t}_{R 2} > {t}_{R 1}$$

t_{R1}, t_{R2}—retention times of the peaks
Wₕ₁, Wₕ₂—peak widths at half-height

The term “full AAV capsids” means that capsids are loaded with a sufficient amount of a vector genome to provide therapeutical efficacy.

The term “empty AAV capsids” means that capsids lack sufficient vector genome and are therefore unable to provide a therapeutic benefit.

When a reference is made to temperature or no temperature is mentioned, the temperature is room temperature (23° C.).

When a reference is made to a volume and no temperature is specified, it is at room temperature.

According to the method of the invention full Adeno-Associated Virus (AAV) capsids are separated from empty AAV capsids by means of chromatography employing a strong anion exchanger material contacting the mixture to be separated. After contacting the mixture, under suitable conditions which are known to the skilled person, an elution of the mixture from the strong anion exchange material is performed by means of a neutral to alkaline buffer comprising an organic modifier with a relative polarity measure of solvent from 0.4 to 0.8. During the elution step in particular the empty AAV capsids separate from the full AAV capsids and can be collected in a fraction separated from other components of the mixture, in particular empty AAV capsids.

Table 1 lists RPM values of various organic compounds. Table 1 enumerates typical organic modifiers to be employed in the method of the invention as acetonitrile, 1-butanol, t-butanol, propylene carbonate, isopropanol, ethanol, methanol or propanol or mixtures thereof.

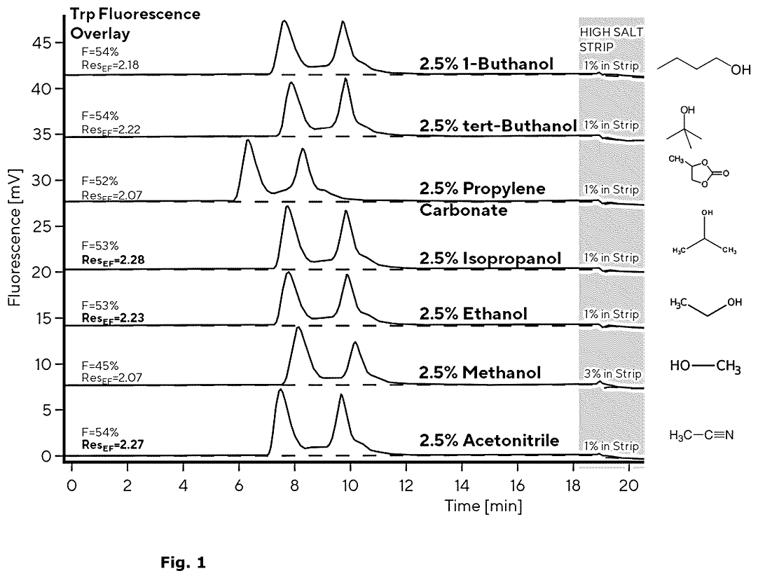

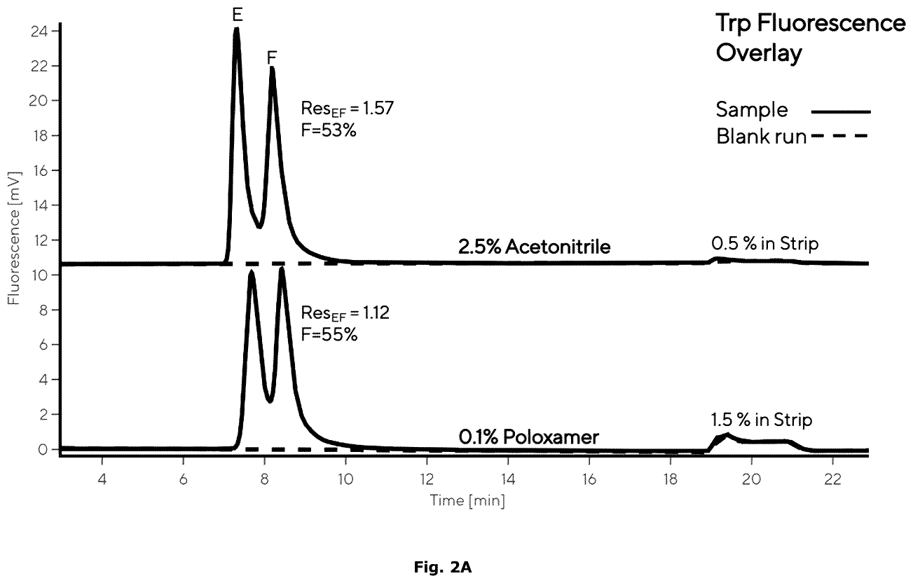

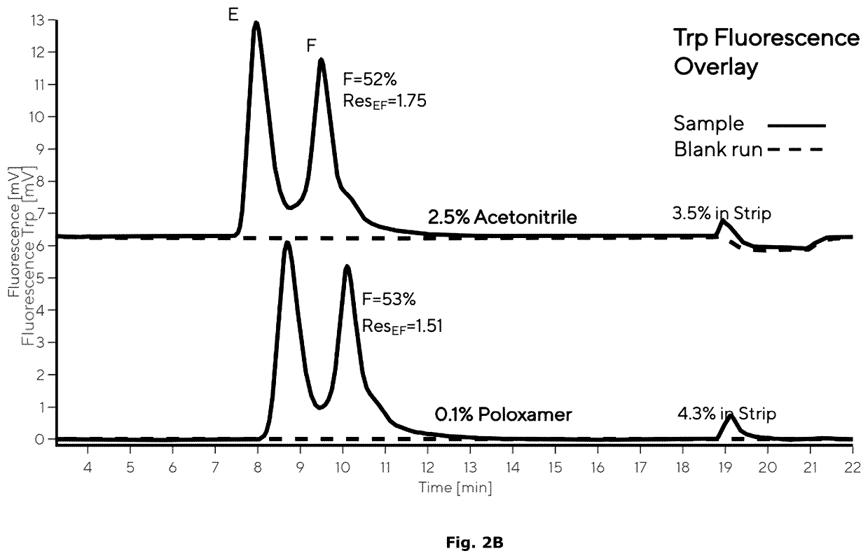

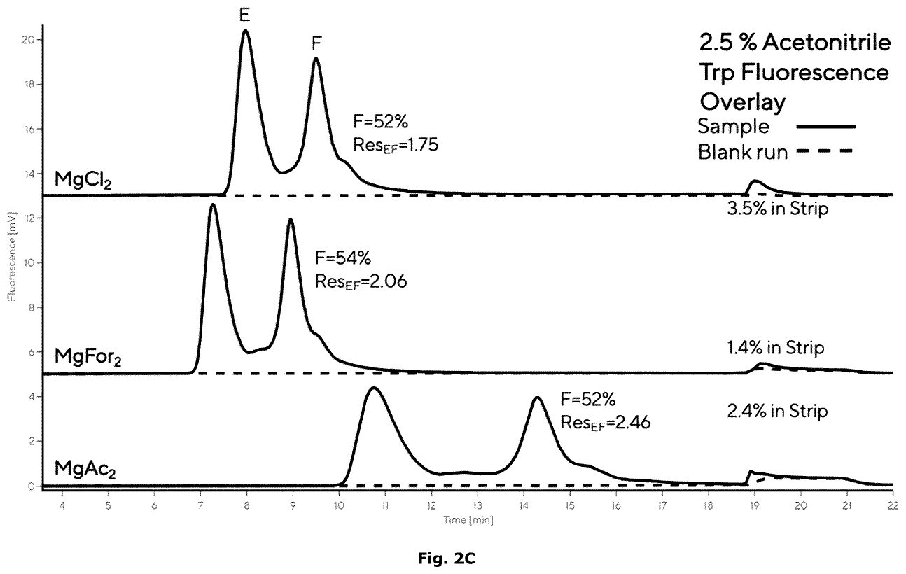

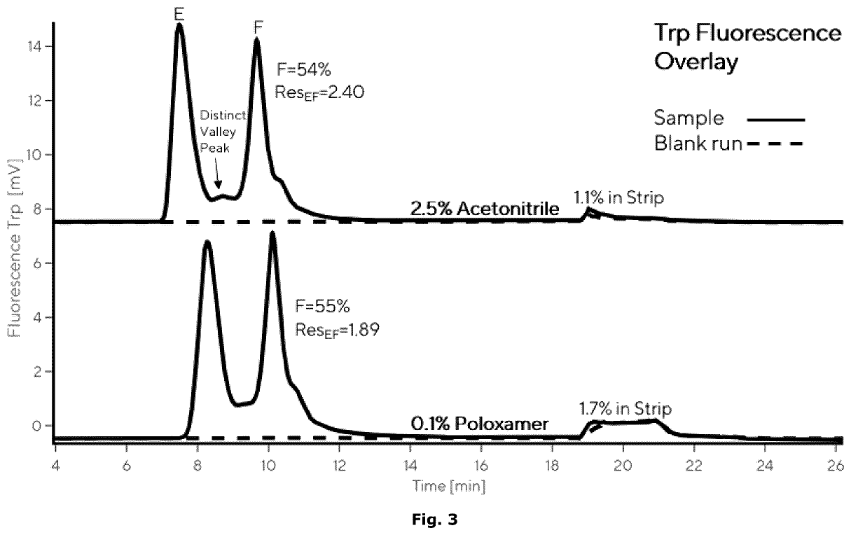

FIG. 1 summarizes the results obtained if typical organic modifiers are used. In the series of experiments underlying the results as depicted in FIG. 2 and FIG. 3 a sample of a mixture containing empty AAV and full AAV capsids, besides other contaminants, was subjected to a monolithic QA anion exchanger in buffers with different organic modifiers. These buffers contain TRIS as buffering agent, sorbitol for stabilisation of capsids, magnesium acetate as eluting salt and different organic modifiers. In standard buffers instead of organic modifiers, poloxamer 188 as a non-ionic surfactant was used.

The separation of empty AAV capsids from full AAV capsids improves significantly if the organic modifiers according to the invention are used compared to the elution buffer avoiding organic modifiers. Poloxamer 188 which is rather an organic compound but outside the range of RPM of 0.4 to 0.8, exhibits a resolution of only 1.89 as depicted in FIG. 3, whereas the resolutions obtained by using the organic modifiers according to the invention are in the range of 2.00 and 2.40.

The results of the experiment shown in FIG. 3 corroborate the effect of improved resolution of separation of full/empty AAV capsids in the presence of at least one organic modifier according to the invention. The use of acetonitrile as a sole organic modifier in presence of magnesium acetate in the elution buffer results in a resolution of 2.40, whereas a buffer containing an organic component poloxamer 188 yields a resolution of only 1.89, significantly less.

It is advantageous that the elution buffer contains not only organic modifier but also alkaline earth metal salts in particular salts of magnesium or calcium and mixtures thereof. Typically, the alkaline earth metal salts are magnesium acetate and/or calcium acetate and/or magnesium or calcium formate. The results of the experiment as depicted in FIG. 2A and FIG. 3 show that the presence of earth alkaline metal salts in elution buffers, e. g. magnesium acetate in conventional concentrations, improve the resolution of the separation of full/empty AAV capsids, compared to the cases in the absence of earth alkaline metal salts. In the experiment of FIG. 2A potassium acetate instead of magnesium acetate (FIG. 3) was present. The resolution of the separation was 1.57 in case of FIG. 2A whereas 2.40 in case of FIG. 3. In the experiment of FIG. 2C the resolution of separation was increased from 1.75 to 2.06 when inorganic anion magnesium chloride was changed to organic magnesium formates. Organic anions with two carbon atoms (or more if soluble in water) enhanced the resolution of separation. The resolution of magnesium formates (FIG. 2C) was enhanced compared to magnesium chloride (FIG. 2C), both with the addition of acetonitrile as organic modifier.

In another example the effect of acetonitrile as the sole organic modifier is depicted in FIG. 2B. The resolution between full and empty AAV capsids is increased from 1.51 in the presence of 188 Poloxamer, to 1.75 in the presence of acetonitrile.

The results depicted in FIGS. 2A, 2B and 2C show that when acetonitrile is used as the organic modifier in elution buffers, improves the resolution between empty and full AAV capsids regardless of different types of alkaline earth metal salts (presence of different cations or anions in elution buffer), such as potassium or magnesium acetate or magnesium chloride. The addition of acetonitrile as the organic modifier of the present invention in the elution buffers provides a particularly advantageous effect on the resolution of empty/full AAV capsids.

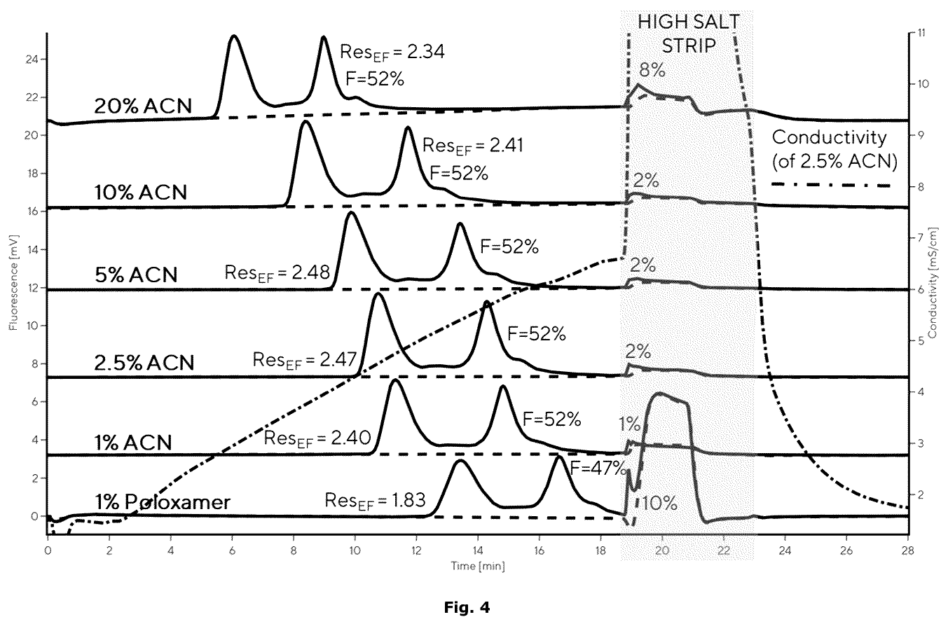

The concentration of the organic modifier in the elution buffer should be as high as necessary and as low as possible. The upper limit of the amount of organic modifier depends on e. g. miscibility of the organic modifier with water and compatibility with other components in the elution buffer. The person skilled in the art is readily able to estimate the range of concentration of the organic modifier. Typically, the organic modifier is present in a range from 1% [volume/volume] to 5% [volume/volume], at concentrations higher than 5% of organic modifier, the resolution between empty and full capsids is lower with the % increase of organic modifier as shown in FIG. 4. Although it seems not very critical to use high concentrations of organic modifier, the skilled person would avoid employing unnecessary high concentrations of organic modifiers.

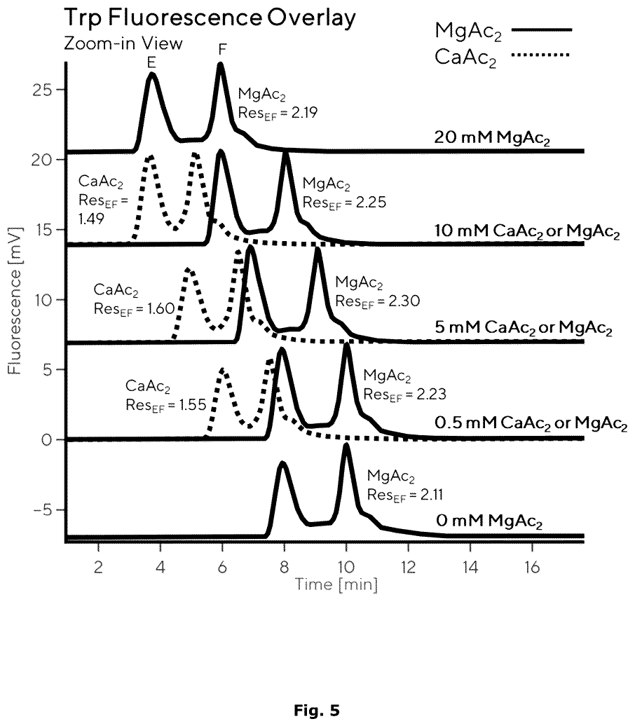

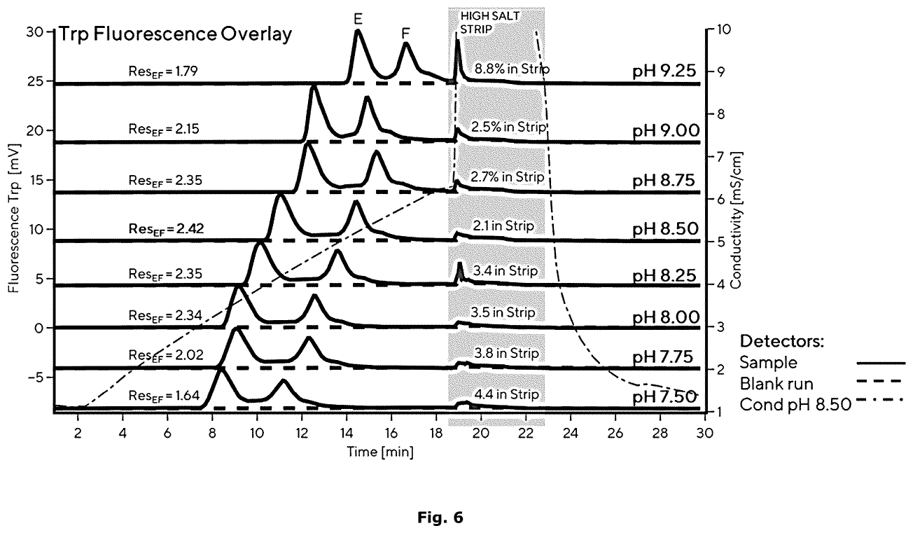

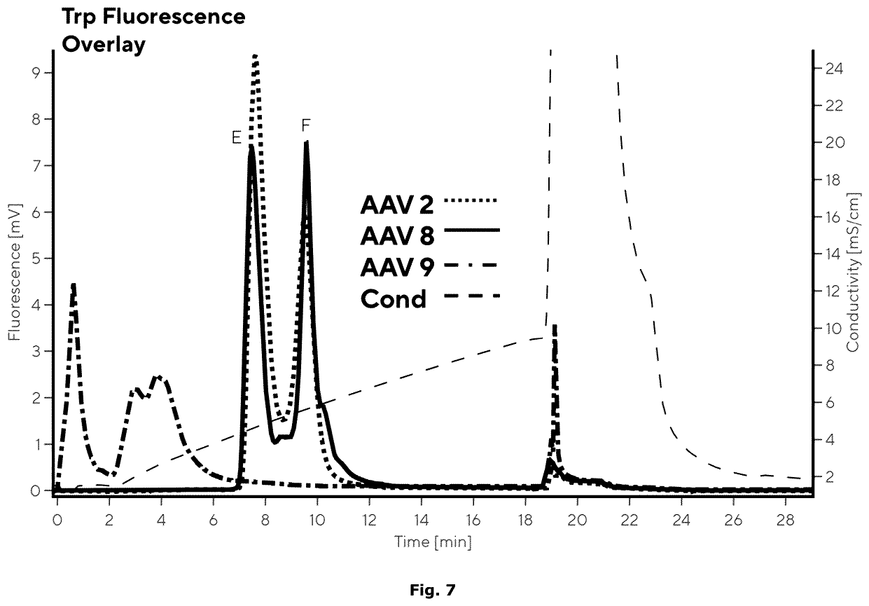

In principle, the person skilled in the art is readily able to adjust proper concentrations of the earth alkaline metal salts in elution buffers used in preparation of AAV capsids. Typically, the concentration range is from about 0.5 mM [weight/volume] to 10 mM [weight/volume]. FIG. 5 shows results obtained when varying the magnesium acetate concentration in the elution buffer. Although it seems not very critical to use high concentrations of magnesium acetate, the skilled person would avoid employing unnecessary high concentrations of magnesium acetate. The resolution is only slightly altered by different magnesium acetate concentrations, although a magnesium acetate concentration of 5 mM seems to be preferable. The pH value of the elution buffer is typically in the range of from about pH 7.5 to about pH 9.25 as shown in FIG. 6. The optimal pH of the buffer depends on the serotype of the respective AAV capsids. An optimal pH value can be evaluated by simple experimentation.

AAV capsids are selected for example from the serotypes AAV1, AAV2, AAV3, AAV4, AAV5, AAV6, AAV7, AAV8, AAV9, AAVrh.10, AAV11, AAV12 and of different serotypes such as hybrid serotypes, chimeras, surface modified AAVs and any synthetic derived AAV like particles. The AAV serotype analysed by the method of invention may be a recombinant hybrid serotype like AAV2/8 or another hybrid serotype.

For example, the separation of full and empty AAV8 serotype capsids is typically performed at a pH of about 8.5, whereas the separation of full and empty AAV9 capsids require higher pH values for optimum separation as shown in FIG. 7.

The buffer substances for adjusting the proper pH value for a separation are able to provide a buffer capacity in an aqueous solution in a range from pH 7 to pH 12, Typically, they are selected from the group consisting of 2-[bis(2-hydroxyethyl)amino]-2-(hydroxymethyl)propane-1,3-diol (Bis-Tris), 2,2′,2″-Nitrilotriacetic acid (ADA), 2-[(2-Amino-2-oxoethyl)amino]ethane-1-sulfonic acid (ACES), 2,2′-(Piperazine-1,4-diyl)di(ethane-1-sulfonic acid) (PIPES), 2-Hydroxy-3-(morpholin-4-yl)propane-1-sulfonic acid (MOPSO), 2,2′-[Propane-1,3-diylbis(azanediyl)]bis[2-(hydroxymethyl)propane-1,3-diol](BTP), N,N-Bis(2-hydroxyethyl)-2-aminoethanesulfonic acid (BES), 3-(Morpholin-4-yl)propane-1-sulfonic acid (MOPS), 2-{[1,3-Dihydroxy-2-(hydroxymethyl)propan-2-yl]amino}ethane-1-sulfonic acid (TES), 2-[4-(2-Hydroxyethyl)piperazin-1-yl]ethane-1-sulfonic acid (HEPES), 3-[N,N-Bis(2-Hydroxyethylamino)-2-Hydroxy-1-Propanesulfonic Acid (DIPSO), 4-(4-Morpholinyl)butanesulfonic acid (MOBS), 2-Hydroxy-3-[tris(hydroxymethyl)methylamino]-1-propanesulfonic acid (TAPSO), 2-Amino-2-(hydroxymethyl)-1,3-propandiol (Trizma), 4-(2-hydroxyethyl)piperazine-1-(2-hydroxypropane-3-sulfonic acid) (HEPPSO), Piperazine-N,N′-bis(2-hydroxypropanesulfonic acid), (POPSO), Triethylamine (TEA), 4-(2-Hydroxyethyl)-1-piperazine-propanesulfonic acid (EPPS), N-tris(hydroxymethyl)methylglycine (Tricine), N,N-Bis(2-hydroxyethyl)glycine (Bicine), N-(2-Hydroxyethyl)piperazine-N′-(4-butanesulfonic acid) (HEPBS), N-tris(Hydroxymethyl)methyl-4-aminobutanesulfonic acid (TAPS), 2-Amino-2-methyl-1,3-propanediol (AMPD), N-tris(Hydroxymethyl)methyl-4-aminobutanesulfonic acid (TABS), N-(1,1-Dimethyl-2-hydroxyethyl)-3-amino-2-hydroxypropanesulfonic acid (AMPSO), 2-(N-Cyclohexylamino)ethanesulfonic acid (CHES), 3-Cyclohexylamino-2-hydroxypropanesulfonic acid sodium salt (CAPSO), N-(1,1-Dimethyl-2-hydroxyethyl)-3-amino-2-hydroxypropanesulfonic acid (AMPS), 3-(Cyclohexylamino)-1-propanesulfonic acid (CAPS) and 4-[Cyclohexylamino]-1-butanesulfonic acid (CABS).

In the elution buffer isotonic substances can be present. Typically, they are selected from the group consisting of sucrose, sorbitol, mannitol, and xylitol.

The buffer may also comprise non-ionic surfactants. In particular so called poloxamers, such as poloxamer 188 can be used. Poloxamers are non-ionic triblock copolymers composed of a central hydrophobic chain of polyoxypropylene (poly(propylene oxide)) flanked by two hydrophilic chains of polyoxyethylene (poly(ethylene oxide)), [U.S. Pat. No. 3,740,421 A]. Poloxamers are also known by the trade names Pluronic®.

The strong anion exchanger material comprises a quaternary amine ligand with commercial names such as Q, QA, QAE, QAM, TEAE, TMAM or TMAE maintaining consistent charge over the range of about pH 2 to pH 13. Quaternary amine anion exchanger materials are disclosed in [10] for separation of empty and full capsid.

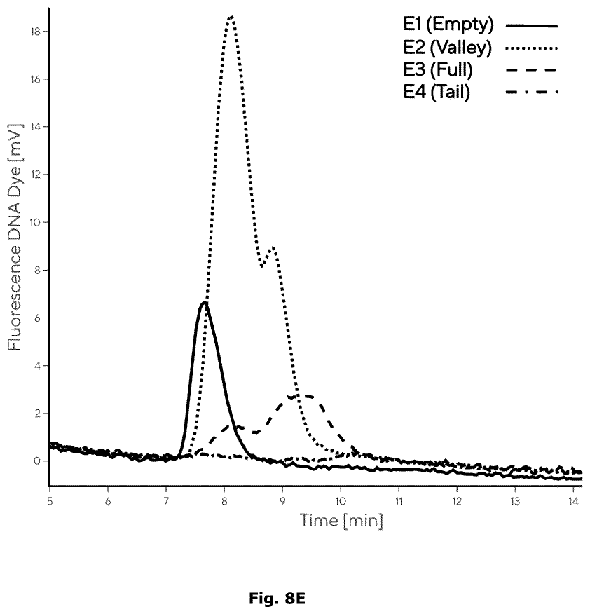

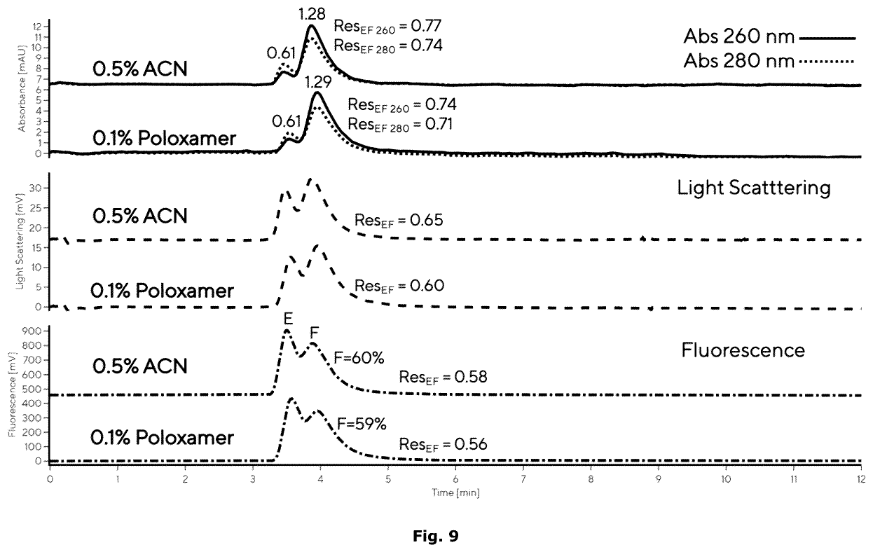

The weak anion exchanger DEAE (diethylaminoethyl) material comprises a tertiary amine ligand with a pKₐ of about 11.5 and allows elution of empty and full capsid at moderate pH values as shown in FIG. 9. [11].

The material may be a monolith anion exchanger material, a particulate anion exchanger material, and/or an anion exchanger material arranged in membranes, and/or particle packed anion exchanger columns and/or fibre chromatography anion exchanger or multimodal fibre columns.

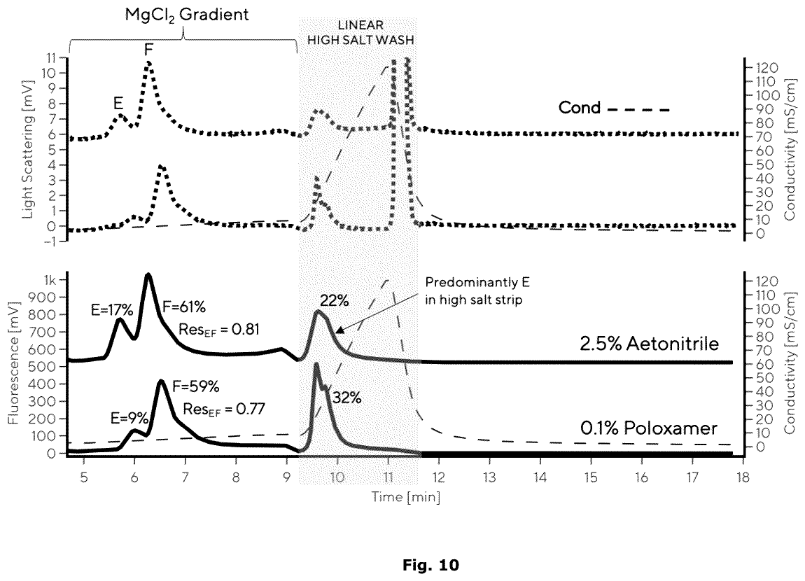

The material may be a multimodal metal affinity exchanger material. That material compromises properties of positively charged metal affinity ligand and weak anion exchanger with hydrogen bond properties [6, 12]. It enables separation of a subpopulation of empty capsids first followed by full capsids in a linear magnesium chloride gradient and later in a high salt step where mostly empty capsids elute as shown in FIG. 10.

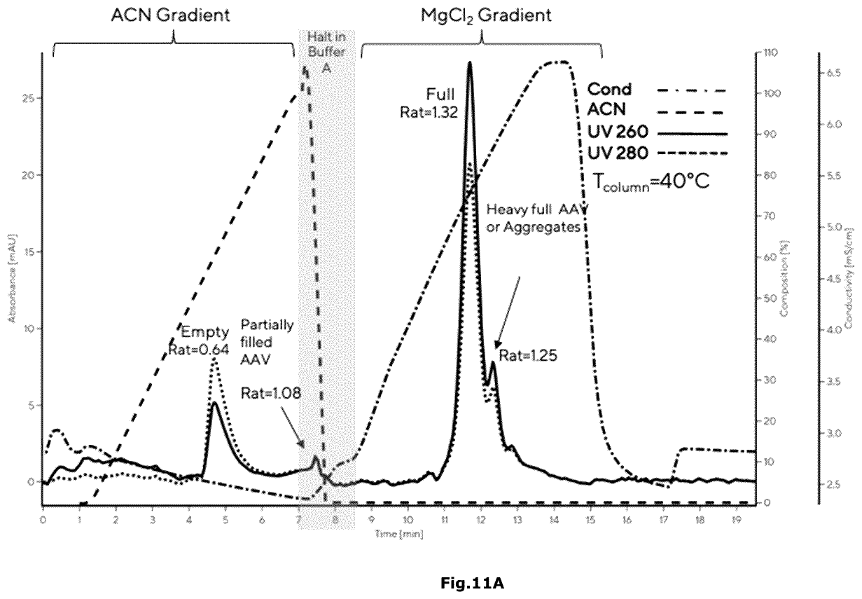

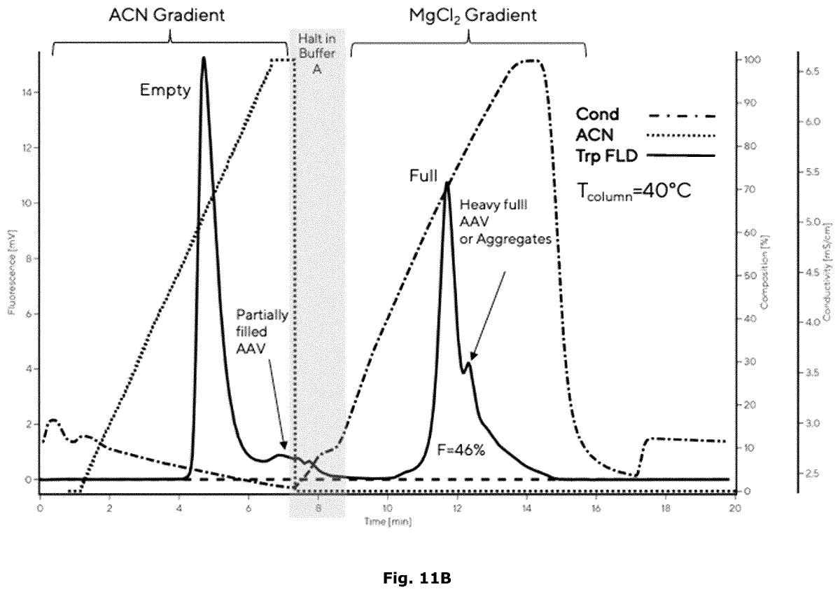

The influence of a higher percentage of organic modifier on AAV capsids separation, particularly on the separation of empty and partially filled capsids using strong anion exchanger is shown in FIGS. 11A and B.

The method of the invention was additionally tested with several loading and elution combination e.g., loading in different salt as the elution salt (e.g., loading in more/less kosmotropic salt), loading in different organic modifier as the elution organic modifier.

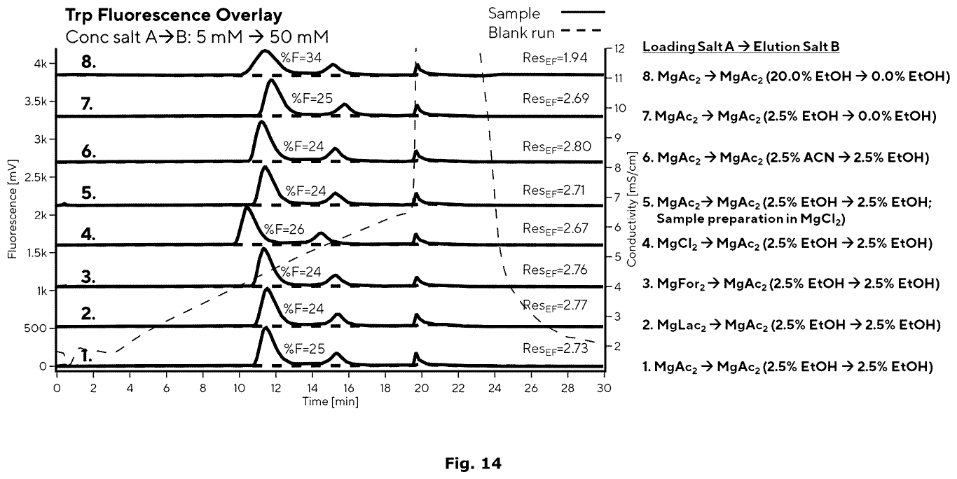

Following both options, a mixed salt or organic modifier gradient was implied in elution gradient (FIG. 14).

In addition, different sample pretreatment (sample pretreated in different salt or eventually with different organic modifier) and decreasing organic modifier gradient (from 2.5% to 0.0% or from 20.0% to 0.0%) were also tested (FIG. 14).

The method of invention is applicable also for analysis of different AAV serotype harvest or lysate samples in one or better in two-dimensional chromatography. Two-dimensional chromatographic system, PATfix™ AAV Switcher (Sartorius BIA Separations, Ajdovscina, Slovenia) enabled analysis of complex crude samples in the upstream of process development.

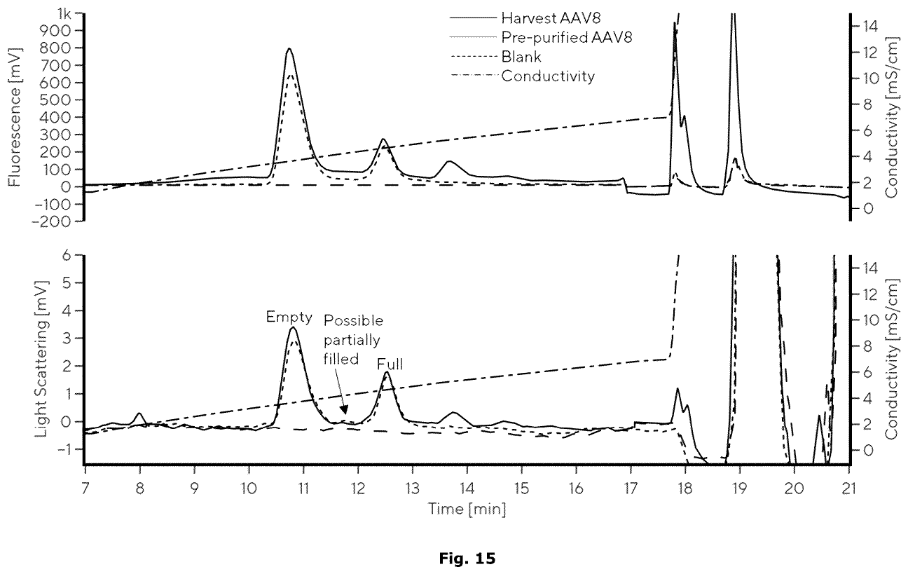

The first column was served for pre-purification of sample and was a strong cation exchange (CEX) and the second column was anion exchange (AEX) column where empty, partially filled, full or other capsid separation was achieved as shown in FIG. 15.

### EXAMPLES

### Example 1

### Preparation of AAV Capsids

The rAAV2/8 was generated through triple transfection of suspension HEK293 cell line in chemically defined media. Rep2-Cap8 and Helper plasmids were used together with cis construct containing GFP expression cassette flanked by inverted terminal repeats (ITRs) regions from AAV2. Plasmids were combined in molar ratio 1:1:1 and transfected to cells using PEI MAX transfection reagent (Polysciences). Transfection was performed in 5 L stirred-tank Biostat B-DCU bioreactor (Sartorius) in fed-batch mode. Cell lysis was performed 72 h post-transfection by adding Tween20 (Sigma-Aldrich) detergent directly into bioreactor. Material was harvested and frozen at −80° C. until further use. Lysed harvest of AAV 2/8 serotype was clarified and then processed by a TFF pre-capture step coupled with DNase treatment. The sample was captured and additionally purified using cation exchange chromatography column-CIMmultus™ SO₃ (Sartorius BIA Separations). Capture step eluate was concentrated and enriched for full AAV capsids using an anion exchange CIMmultus™ QA column. Finally, separately collected empty and full AAV capsid containing fractions were buffer exchanged into formulation buffer using the Vivaspin Turbo 100 kDa PES concentrator. A sample prepared with a pool of empty and full fractions in ratio 1:2 and 2:1 was used for all analytical runs and preparative runs respectively.

### Example 2

2a—Effect of Different Organic Modifiers on the Resolution of Empty/Full AAV Capsids

Analytical separations of empty and full AAV capsid samples were performed on a 100 μL strong anion exchanger, CIMac™ AAV full/empty column. The column was equilibrated with 2 mM magnesium acetate, 2.5% organic modifier, 20 mM TRIS and 1% sorbitol at pH 8.5 and eluted with linear salt gradient to 80 mM magnesium acetate, 2.5% organic modifier, 20 mM TRIS and 1% sorbitol at pH 8.5. The volumetric flow rate was 1 mL/min. As a strip buffer 2000 mM potassium acetate, 2.5% organic modifier, 20 mM TRIS and 1% sorbitol at pH 8.5 was used.

Screening of some easily accessible organic modifiers is presented in FIG. 1 and the highest resolution of around 2.28 of empty and full AAV capsids is provided by isopropanol, acetonitrile followed by ethanol, tert- and 1-butanol as shown in FIG. 1. Other tested organic solvents deliver lower resolution and thus are less preferrable e.g., propylene carbonate and methanol.

In addition to the empty and full AAV capsid separation all tested organic modifiers indicate subpopulation separation.

2b—Effect of Replacing Conventionally Used Poloxamer 188 for Organic Modifier and Effect of Different Loading and Elution Salts on the Resolution of Empty/Full AAV Capsids Analytical separations of empty and full AAV capsid samples were performed on a 100 μL strong anion exchanger, CIMac™ AAV full/empty column. The column was equilibrated and eluted with:
For FIG. 2A: equilibrated with 2 mM magnesium chloride, 20 mM BTP, 1% sorbitol, 2.5% acetonitrile or Poloxamer 188 at pH 9.3 and eluted with linear salt gradient to 2 mM magnesium chloride, 400 mM potassium acetate, 20 mM BTP, 1% sorbitol, 2.5% acetonitrile or Poloxamer 188 at pH 9.3. The volumetric flow rate was 1 mL/min. As a strip buffer 2000 mM potassium acetate, 2.5% acetonitrile or 0.1% Poloxamer 188, 20 mM BTP at pH 9.3 was used.
For FIG. 2B: equilibrated with 2 mM magnesium acetate, 2.5% acetonitrile or 0.1% Poloxamer 188, 20 mM TRIS and 1% sorbitol at pH 8.5 and eluted with linear salt gradient to 50 mM magnesium acetate, 2.5% acetonitrile or 0.1% Poloxamer 188, 20 mM TRIS and 1% sorbitol at pH 8.5. The volumetric flow rate was 1 mL/min. As a strip buffer 2000 mM potassium acetate, 2.5% acetonitrile or 0.1% Poloxamer 188, 20 mM TRIS at pH 8.5 was used.

In FIGS. 2A and 2B comparison between conventionally used Poloxamer 188 and acetonitrile as organic modifier is presented. In both experiments regardless of the used salt the resolution was improved for approximately 20-40% only by swapping Poloxamer 188 for acetonitrile. Moreover, percentage of AAV capsids in high salt strip was in both cases (FIGS. 2A and 2B) lower for acetonitrile.
For FIG. 2C: equilibrated with 2 mM magnesium acetate/formate/chloride, 2.5% acetonitrile, 20 mM TRIS and 1% sorbitol at pH 8.5 and eluted with linear salt gradient to 50 mM magnesium acetate/chloride (80 mM in case of magnesium formate), 2.5% acetonitrile, 20 mM TRIS and 1% sorbitol at pH 8.5. The volumetric flow rate was 1 mL/min. As a strip buffer 2000 mM potassium acetate, 2.5% acetonitrile and 20 mM TRIS at pH 8.5 was used.

From the FIG. 2C it is evident that magnesium acetate shows best resolution, compared to formate and especially compared to tested inorganic chloride.

2c—Effect of Organic Modifier as a Sole Compared to Poloxamer 188 on the Resolution of Empty/Full AAV Capsids

Analytical separations of empty and full AAV capsid samples were performed on a 100 μL strong anion exchanger, CIMac™ AAV full/empty column. The column was equilibrated with 2 mM magnesium acetate, 2.5% acetonitrile, 20 mM TRIS and 1% sorbitol at pH 8.5 and eluted with linear salt gradient to 80 mM magnesium acetate, 2.5% acetonitrile, 20 mM TRIS and 1% sorbitol at pH 8.5. The volumetric flow rate was 1 mL/min. As a strip buffer 2000 mM potassium acetate, 2.5% acetonitrile, 20 mM TRIS and 1% sorbitol at pH 8.5 was used.

Corresponding buffer combination provides the high resolution of 2.40 of empty and full AAV capsids as shown in FIG. 3 compared to the resolution of 1.89 when poloxamer 188 was used. The combination of Poloxamer 188 and organic modifiers e.g., acetonitrile would be lower compared to the acetonitrile as a sole. The elution was achieved in a linear salt gradient from 2 mM to 80 mM magnesium acetate with 160 column volume (CV). In addition to the empty and full AAV capsid separation the buffer combination indicates subpopulation separation.

2d—Effect of Different Percentage of Organic Modifiers on the Resolution of Empty/Full AAV Capsids

Analytical separations of empty and full AAV capsid samples were performed on a 100 μL strong anion exchanger, CIMac™ AAV full/empty column. The column was equilibrated with 2 mM magnesium acetate, X % acetonitrile, 20 mM TRIS and 1% sorbitol at pH 8.5 and eluted with linear salt gradient to 50 mM magnesium acetate, X % acetonitrile, 20 mM TRIS and 1% sorbitol at pH 8.5. The volumetric flow rate was 1 mL/min. As a strip buffer 2000 mM potassium acetate, X % acetonitrile, 20 mM TRIS and 1% sorbitol at pH 8.5 was used. For comparison 1% Pooxamer 188 was used instead of acetonitrile.
X: 1, 2.5, 5, 10, 20% Acetonitrile

Best results are shown to be with 5% and 2.5% of acetonitrile (highest resolution between empty and full AAV8 and lowest percentage of high salt strip) as shown in FIG. 4. However, the lowest possible percentage of the organic modifier e.g., acetonitrile is preferred due to the possible scale-up of the method to preparative scale and safety reasons. With higher or lower percentage of acetonitrile the resolution drops. With 1% Poloxamer 188 the resolution was the lowest and the percentage of AAV in high salt strip was the highest (FIG. 4).

2e—Effect of Different Magnesium or Calcium Acetate Loading Concentrations on the Resolution of Empty/Full AAV Capsids

Analytical separations of empty and full AAV capsid samples were performed on a 100 μL strong anion exchanger, CIMac™ AAV full/empty column. The column was equilibrated with X mM magnesium or calcium acetate, 2.5% acetonitrile, 20 mM TRIS and 1% sorbitol at pH 8.5 and eluted with linear salt gradient to Y mM magnesium or calcium acetate, 2.5% acetonitrile, 20 mM TRIS and 1% sorbitol at pH 8.5. The volumetric flow rate was 1 mL/min. As a strip buffer 2000 mM potassium acetate, 2.5% acetonitrile, 20 mM TRIS and 1% sorbitol at pH 8.5 was used.
X: 0, 0.5, 5, 10, 20 mM magnesium or calcium acetate
Y: concentration of elution magnesium or calcium acetate was adjusted to reach the same gradient speed as the standard 2-80 mM magnesium acetate method.

In general calcium acetate is less preferrable than magnesium acetate (FIG. 5) which delivers significantly better resolutions. Furthermore, the preferred concentration of a loading magnesium acetate is around 5 mM. Concentrations of 0.5 and 10 mM of magnesium acetate deliver slightly worse results.

2f—Effect of Different pH Values on the Resolution of Empty/Full AAV Capsids

Analytical separations of empty and full AAV capsid samples were performed on a 100 μL strong anion exchanger, CIMac™ AAV full/empty column. The column was equilibrated with 2 mM magnesium acetate, 2.5% ethanol, 20 mM TRIS and 1% sorbitol at pH 7.50-9.25 and eluted with linear salt gradient to 50 mM magnesium acetate, 2.5% ethanol, 20 mM TRIS and 1% sorbitol at pH 7.50-9.25. The volumetric flow rate was 1 mL/min. As a strip buffer 2000 mM potassium acetate, 2.5% ethanol, 20 mM TRIS and 1% sorbitol at pH 7.50-9.25 was used. The highest resolution between empty and full AAV8 capsids and the lowest percentage of high salt strip is enabled with buffers at pH 8.50 as depicted in FIG. 6. Other pH values (especially below 8.00 and above 8.75) deliver worse results.

2g—Extension of the Method to Other AAV Serotypes

Analytical separations of empty and full AAV capsid samples were performed on a 100 μL strong anion exchanger, CIMac™ AAV full/empty column. The column was equilibrated and eluted with:
2 mM magnesium acetate, 2.5% acetonitrile, 20 mM TRIS and 1% sorbitol at pH 8.50 and eluted with linear salt gradient to 80 mM magnesium acetate, 2.5% acetonitrile, 20 mM TRIS and 1% sorbitol at pH 8.50. The volumetric flow rate was 1 mL/min. As a strip buffer 2000 mM potassium acetate, 2.5% acetonitrile, 20 mM TRIS and 1% sorbitol at pH 8.50 was used.

The method of the invention was also tested for AAV2 and AAV9 serotype (FIG. 7). However, it is recommended to modify the method for each serotype to obtain improved resolution.

### Example 3

Preparative Separation with QA

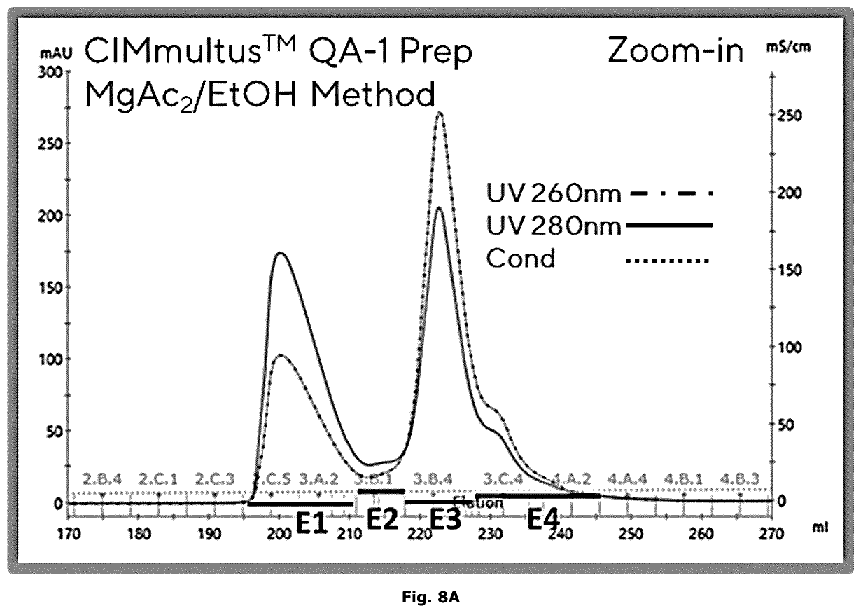

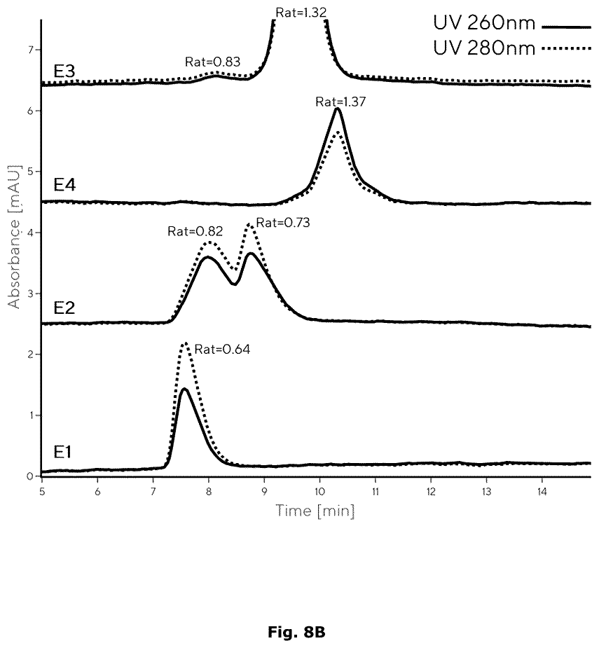

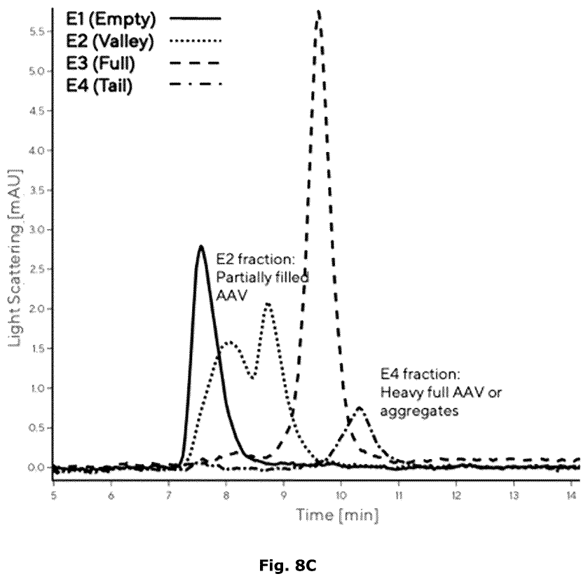

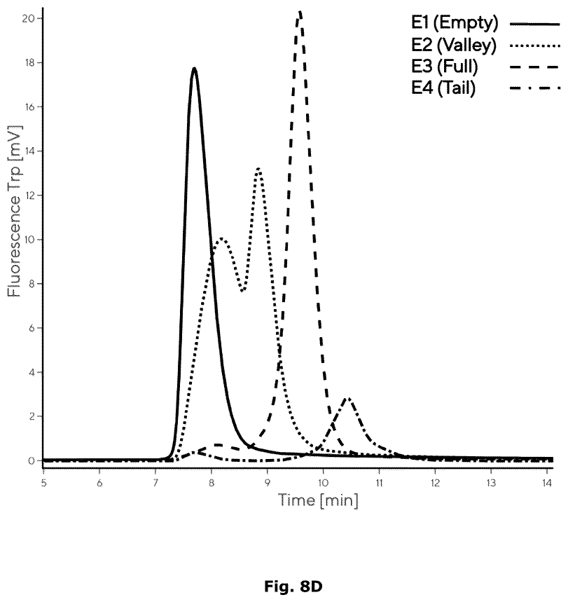

A preparative separation of empty from full AAV capsids was performed by using a CIMmultus™ QA-1 mL (2 μm) column. The buffers and elution conditions of example 2 were employed, except ethanol was used as the organic modifier instead of acetonitrile. Fractions E1 to E4 from preparative run were collected as shown in FIG. 8A. Fractions were individually analyzed on analytical scale using a multidetector setup, UV 260 and 280 nm, light scattering, intrinsic protein fluorescence that mostly induced by tryptophan and extrinsic fluorescence by using intercalating dye. Tryptophan is abundant in AAV capsids proteins, constituting 2.2% of capsids [13,14], fluorescence is measured at extinction 280 nm and emission at 348 nm. Extrinsic fluorescence is measured at extinction 485 nm and emission at 520 nm using intercalating dye PicoGreen to amplify sensitivity of nucleic acids impurities [15]. FIG. 8B shows UV results where, E1 fraction represents empty capsids with 260/280 wavelength ratio of 0.64. E2 fraction was collected between empty and full peak (valley fraction) and shows two populations with 260/280 wavelength ratios 0.82 and 0.73. This fraction highly likely represents partially filled capsids or DNA associated with empty capsid exteriors as resulted from extrinsic PicoGreen fluorescence. E3 fraction is populated with full capsids with 260/280 wavelength ratio of 1.37. Fraction E4 (tail fraction) highly likely represents damaged AAV capsids and/or aggregates. Elution fractions monitored by light scattering detector are shown in FIG. 8C. Elution fractions monitored by tryptophan fluorescence are shown in FIG. 8D and elution fractions monitored byPicoGreen fluorescence are shown in FIG. 8E. The high PicoGreen fluorescence signal of the E2 fraction (FIG. 8E) indicates considerable amount of DNA related impurities compared to other elution fractions.
Note: UV 260/280 ratio is abbreviated as Rat in FIG. 8B.

### Example 4

### Separation of Empty and Full AAV Capsids Using a Weak Anion Exchanger

The separation of empty and full AAV capsids was performed using a 100 μL CIMac™ DEAE column.

The column was equilibrated with 20 mM TRIS, 0.5% acetonitrile or 1% poloxamer 188, 1% sorbitol at pH 9.0 eluted with a linear salt gradient to 50 mM magnesium acetate, 20 mM TRIS, 0.5% acetonitrile or 1% poloxamer 188, 1% sorbitol at pH 9.0. The volumetric flow rate was 1 mL/min. There was no salt added in equilibration buffer due to the weak sample binding. FIG. 9 depicts results showing that resolutions were slightly enhanced when an organic modifier was introduced to the buffers. As a strip buffer 2000 mM potassium acetate, 0.5% acetonitrile or 0.1% Poloxamer, 20 mM TRIS at pH 9.0 was used.

### Example 5

Separation of Empty and Full AAV Capsids with a Multimodal Material

The separation of empty and full AAV capsids was achieved on a 100 μL CIMac™ PrimaT column which was a multimodal metal affinity ligand that combines elements of hydrogen with anion exchange chromatography.

Sample binding on the column was achieved at neutral pH due to dominant H-bond, with 25 mM HEPES, 1% saccharose, 0.1% poloxamer 188 or 2.5% acetonitrile buffer; pH transition was performed with 50 mM Tris, 13.6 mM borate, 1% saccharose, 0.1% poloxamer 188 or 2.5% acetonitrile at pH 9.0 to affect electrostatic interactions over H-bond. The majority of full and some of empty AAV capsids were eluted with linear salt gradient to 50 mM magnesium chloride, 50 mM Tris, 9.6 mM borate, 1% saccharose, 0.1% poloxamer 188 or 2.5% acetonitrile at pH 9.0 following by high salt linear gradient to 2 M NaCl, 50 mM Tris, 12 mM borate, 1% saccharose, 0.1% poloxamer 188 or 2.5% acetonitrile at pH 9.0 where elution of the remaining empty AAV capsids was achieved. Volumetric flow rate was 1 mL/min.

When poloxamer 188 was replaced by acetonitrile an enhanced resolution was obtained and less empty AAV capsids in high salt linear gradient was observed [FIG. 10].

### Example 6

### Influence of Concentration of Organic Modifier

The influence of a higher percentage of organic modifier in combination with a salt gradient on AAV capsids baseline separation was investigated by using a CIMac™ QA column. The column was equilibrated with 10 mM magnesium acetate, 50 mM TRIS, 2% acetonitrile, 1% sorbitol at pH 8.5 (buffer A), eluted first with linear acetonitrile gradient to 30%, 10 mM magnesium acetate, 50 mM TRIS, 1% sorbitol at pH 8.5. After employing the acetonitrile gradient the column was halted with buffer A to lower percentage of acetonitrile and eluted with a salt gradient to 50 mM magnesium acetate, 50 mM TRIS, 1% sorbitol at pH 8.5. The volumetric flow rate was 1 mL/min.

Elution conditions using higher concentrations of organic modifier followed by employing a salt gradient enabled baseline separation of empty and full AAV capsids from other species. In FIG. 11A UV 260 and 280 nm chromatogram depicts separation of AAV capsids achieved by combination of reverse phase conditions (elution of empty with 260/280 wavelength ratio 0.64 and partially filled AAV capsids with 260/280 ratio 1.08) followed by anion exchange conditions elution of full capsids with 260/280 ratio 1.32 as dominant peak followed with tailing peak with 260/280 ratio 1.25. A higher temperature, 40° C. and combination of reverse phase and anion exchange conditions allowed baseline separation of empty and partially filled AAV capsids from other subpopulations of full AAV capsids, in particular of full and heavy full AAV capsids or aggregates. The same trend was observed with tryptophan fluorescence chromatogram in FIG. 11B.

### Example 7

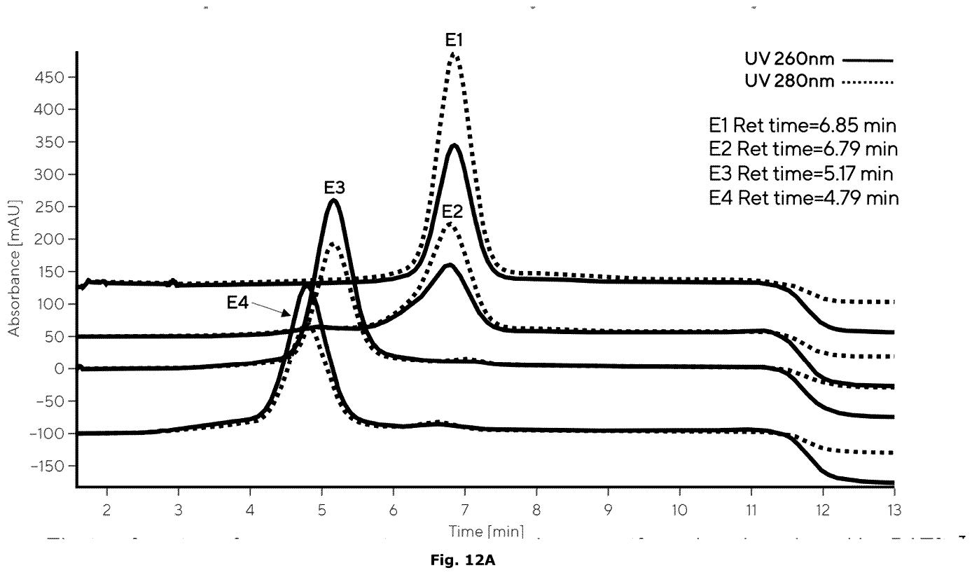

Orthogonal analysis of preparative fractions using density gradient ultracentrifugation coupled with PATfix™ multiple detectors array [19]. During density gradient ultracentrifugation, capsid populations are separated based on their density, with full capsids at the bottom and empty capsids at the top of the CsCl gradient. After ultracentrifugation fractions were pumped through for analysis of UV 260 and 280 nm signals (FIG. 12A) and tryptophan intrinsic fluorescence (FIG. 12B). Results of UV 260 and 280 nm signal of E1 fraction (FIG. 12A) eluted at 6.85 min corresponded to empty AAV capsids. In E2 fraction, partly filled AAV capsids were observed at 6.78 min. Distinct fronting peak of E2 fraction from ~5.5 to 6.5 min indicated presence of slightly heavier capsids than empty capsids. E3 fraction at 5.17 min showed mainly full capsids. E4 fraction eluted earlier compared to E3 fraction and showed fronting peak from 3 to 4 min which indicated presence of heavier AAV capsids or aggregates.

Density ultracentrifugation results obtained supports analytical and preparative results.

### Example 8

### Comparison of AAV Capsids Separation Using QA Column and Anion Exchanger Membrane Adsorber

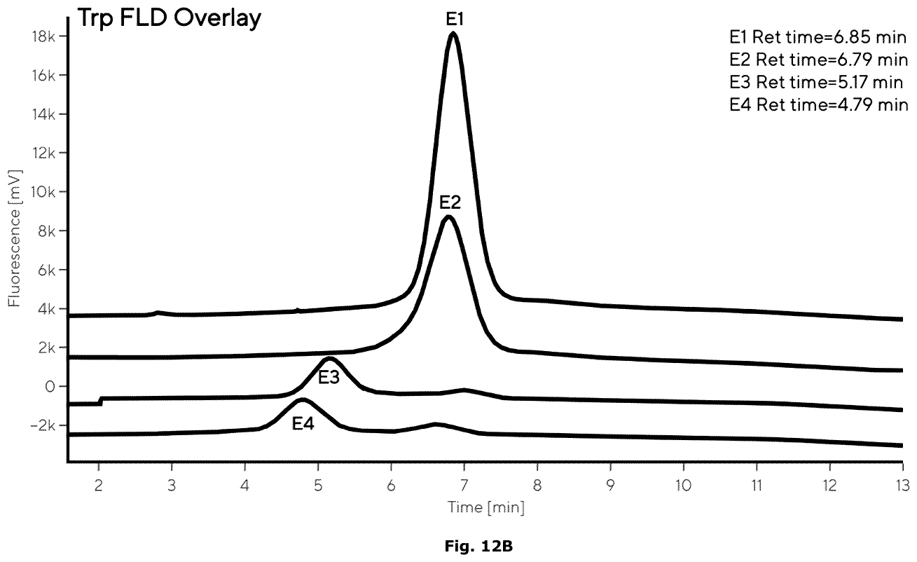

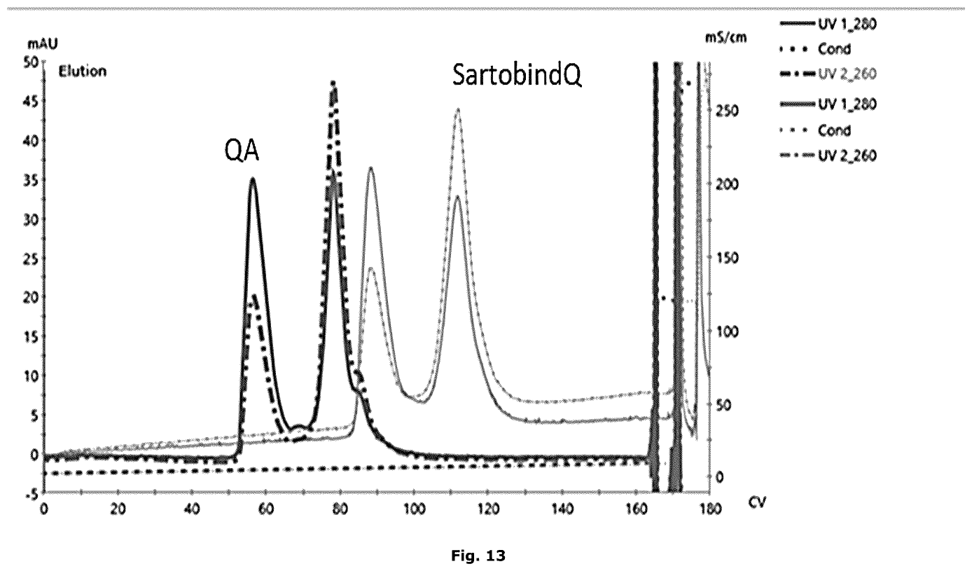

A preparative run with sample material described in example 1 was loaded on a monolith anion exchange column and a membrane adsorber Sartobind®Q—3 mL column (Sartorius). The buffers and elution conditions of example 3 were employed. Similar separation profiles were obtained by both QA column and membrane adsorber as shown in FIG. 13.

### Example 9

### Preparation of AAV Capsids

The rAAV2/8 was generated through triple transfection of suspension HEK293 cell line in chemically defined media. Rep2-Cap8 and Helper plasmids were used together with cis construct containing GFP expression cassette flanked by inverted terminal repeats (ITRs) regions from AAV2. Plasmids were combined in molar ratio 1:1:1 and transfected to cells using PEI MAX transfection reagent (Polysciences). Transfection was performed in 5 L stirred-tank Biostat B-DCU bioreactor (Sartorius) in fed-batch mode. Cell lysis was performed 72 h post-transfection by adding Tween20 (Sigma-Aldrich) detergent directly into bioreactor. Material was harvested and frozen at −80° C. until further use. Lysed harvest (used for Example 2 experiment) of AAV 2/8 serotype was clarified and then processed by a TFF pre-capture step coupled with DNase treatment. The sample was captured and additionally purified using cation exchange chromatography column-CIMmultus™ SO3 (Sartorius BIA Separations). Capture step eluate was concentrated, buffer exchanged into formulation buffer using the Vivaspin Turbo 100 kDa PES concentrator and used for the experiments. This sample was only used for experiments in example 10 and 11.

### Example 10

### Effect of Different Loading and Elution Variables on the Resolution of Empty/Full AAV Capsids

Analytical separations of empty and full AAV capsid samples were performed on a 100 μL strong anion exchanger, CIMac™ AAV full/empty column. The column was equilibrated with loading buffer (Buffer A) with different magnesium salt or different organic solvent or percent of organic solvent acting as organic modifier. In two cases, percent of organic solvent was 0.0% (decreasing gradient of organic modifier).
Buffer A (loading buffer): X mM magnesium salt, Y % organic modifier, 20 mM TRIS and 1% sorbitol at pH 8.5
Buffer B (elution buffer): 50 mM magnesium acetate, Y % organic modifier, 20 mM TRIS and 1% sorbitol at pH 8.5 Sample was loaded in buffer A and eluted with linear salt gradient to buffer B. The volumetric flow rate was 1 mL/min.
Buffer C was applied to elute more electronegative compounds that stayed bound after elution with buffer B.
Buffer C (high salt wash): 2000 mM potassium acetate, 2.5% ethanol, 20 mM TRIS and at pH 8.5

### 1. Experiment:

Buffer A: 5 mM magnesium acetate, 2.5% ethanol, 20 mM TRIS and 1% sorbitol at pH 8.5
Buffer B: 50 mM magnesium acetate, 2.5% ethanol, 20 mM TRIS and 1% sorbitol at pH 8.5

### 2. Experiment:

Buffer A: 5 mM magnesium lactate, 2.5% ethanol, 20 mM TRIS and 1% sorbitol at pH 8.5
Buffer B: 50 mM magnesium acetate, 2.5% ethanol, 20 mM TRIS and 1% sorbitol at pH 8.5

### 3. Experiment:

Buffer A: 5 mM magnesium formate, 2.5% ethanol, 20 mM TRIS and 1% sorbitol at pH 8.5
Buffer B: 50 mM magnesium acetate, 2.5% ethanol, 20 mM TRIS and 1% sorbitol at pH 8.5

### 4. Experiment:

Buffer A: 5 mM magnesium chloride, 2.5% ethanol, 20 mM TRIS and 1% sorbitol at pH 8.5
Buffer B: 50 mM magnesium acetate, 2.5% ethanol, 20 mM TRIS and 1% sorbitol at pH 8.5

### 5. Experiment:

Buffer A: 5 mM magnesium acetate, 2.5% ethanol, 20 mM TRIS and 1% sorbitol at pH 8.5
Buffer B: 50 mM magnesium acetate, 2.5% ethanol, 20 mM TRIS and 1% sorbitol at pH 8.5

Sample was prepared in 5 mM magnesium chloride, 2.5% ethanol, 20 mM TRIS and 1% sorbitol at pH 8.5.

### 6. Experiment:

Buffer A: 5 mM magnesium acetate, 2.5% acetonitrile, 20 mM TRIS and 1% sorbitol at pH 8.5
Buffer B: 50 mM magnesium acetate, 2.5% ethanol, 20 mM TRIS and 1% sorbitol at pH 8.5

### 7. Experiment:

Buffer A: 5 mM magnesium acetate, 2.5% ethanol, 20 mM TRIS and 1% sorbitol at pH 8.5
Buffer B (no organic modifier): 50 mM magnesium acetate, 20 mM TRIS and 1% sorbitol at pH 8.5

### 8. Experiment:

Buffer A: 5 mM magnesium acetate, 20.0% ethanol, 20 mM TRIS and 1% sorbitol at pH 8.5
Buffer B (no organic modifier): 50 mM magnesium acetate, 20 mM TRIS and 1% sorbitol at pH 8.5

The results above suggests that all the options delivered comparable results with no significant differences between the experiments regarding resolution between empty and full AAV capsids and regarding percentage of full AAV8. The lowest resolution and estimated percentage of full was in case of loading in high percentage and elution to zero percentage of ethanol as shown in FIG. 14, sample 8. Moreover, loading in less kosmotropic salt (magnesium chloride) particularly resulted in lower resolution (sample 4) in FIG. 14. On the other hand, as expected, loading in more kosmotropic salt (magnesium lactate) resulted in slightly higher resolution compared to magnesium acetate (sample 1) in FIG. 14.

When combining more potentially different elution strategies it is beneficial to have robust binding and loading conditions to obtain reproducible results.

### Example 11

### PATfix AAV Switcher (Sartorius BIA Separations, Ajdovscina, Slovenia) of Lysate Sample

The following buffers for AEX separation were used:
Buffer A: 20 Tris+5 mM magnesium acetate+1% Sorbitol+2.5% EtOH; pH 8.50
Buffer B: 20 Tris+65 mM magnesium acetate+1% Sorbitol+2.5% EtOH; pH 8.50
Buffer C: 500 mM sodium acetate; pH 5.50
Buffer D: 100 mM sodium hydroxide+2000 mM sodium chloride

A separation of empty, possible partially filled, full and/or other capsids in com-plex lysate sample was achieved on a CIMac™ QA HR column by using similar conditions as the method described above. The lysate sample was first purified on a cation exchange column using a pH gradient, from pH 4.50 to pH 9.50. The proportion of the pH gradient elution was then in-line redirected on the second AEX column where a multiple subpopulations separation was achieved. In addition to the empty and full capsids also partially filled or other impurities were observed (FIG. 15). Fronting peak from ~8 to ~10 minutes represents some protein impurities present in harvest sample (observed by Tryptophan flu-orescence but not by light scattering). Peak at around 13.7 minutes corre-sponds to other most likely larger protein related impurities present in harvest sample (peak seen by fluorescence and light scattering). Other contaminants eluted in AEX column cleaning step (from ~18 minutes on). One dimensional analytics would also deliver similar results but with higher probability of more impurities from harvest sample coeluting with AAV capsids which would also result in shorter column lifetime due to the highly negatively charged impurities (which may be eliminated by cation exchange (CEX) column in-line pre-purification) staying bound onto AEX column.

TABLE 1

| | [symbol] [a, b] | R [symbol] P [symbol] M [symbol] |
|---|---|---|
| Solvent | [kcal/mol] | [ [symbol] ] |
| 1,1,1,3,3,3-Hexaflouro-2-propanol | 69.3 [d] | −1.243 |
| Water | 63.1 | −1.04 [symbol] |
| [symbol] -Trifluoroethanol | [symbol] 9.5 | −0.926 |
| [symbol] -Tetrafluoro-1-propanol | [symbol] 9.4 | −0.922 |
| 1,2,3-Propanetriol (glycerol) | 57.0 [e] | −0.845 |
| Formamide | [symbol] 6.6 | −0.832 |
| [symbol] 2-Ethandiol (glycol) | [symbol] 6.3 | −0.82 [symbol] |
| Methanol | 55.5 | −0. [symbol] 96 |
| 1,3-Propaned [symbol] | 54. [symbol] [ [symbol] ] | −0.777 |
| 1,2-Propanediol | 54.1 [e] | −0.7 [symbol] |
| N-Methy [symbol] formamide | 54.1 | −0. [symbol] 1 |
| Diethylene glycol | 53.8 | −0. [symbol] 41 |
| Ethanol/water (80:20 vol [symbol] %) | 53.7 | −0.738 |
| Triethylene glycol | [symbol] .5 | −0.7 [symbol] 1 |
| [symbol] -Buta [symbol] l | [symbol] 2.8 [ [symbol] ] | −0.709 |
| 2-Prop [symbol] | [symbol] 2. [symbol] [d] | −0.699 |
| 2-Methoxy [symbol] | [symbol] .3 | −0.693 |
| [symbol] ( [symbol] alcohol) | [symbol] .1 | −0.686 |
| N-Methylace [symbol] mide | [symbol] 2.0 [ [symbol] ] | −0.683 |
| Ethanol | [symbol] 1. [symbol] | −0.680 |
| [symbol] -Amino [symbol] ol | [symbol] 1.8 | −0.676 |
| Acetic acid | [symbol] 1.2 [ [symbol] ] | −0.657 |
| [symbol] alcohol | [symbol] 0. [symbol] | −0. [symbol] |
| 1-Propanol | [symbol] 0. [symbol] | −0.641 |
| 1-Butan [symbol] | [symbol] 0. [symbol] | −0.625 |
| 2-Hydroxymethy [symbol] alcohol) | 50.0 [d] | −0.618 |
| 2-Phen [symbol] ethanol | 49. [symbol] [ [symbol] ] | −0.602 |
| 1-Pen [symbol] | 4 [symbol] .1 | −0. [symbol] 89 |
| 2-Methyl- [symbol] propanol (isobutyl alcohol) | 4 [symbol] .0 | −0.586 |
| 1-Hexanol | [symbol] . [symbol] | −0.579 |
| 2-Propanol | 48.6 | −0. [symbol] 7 [symbol] |
| 3-Phenyl- [symbol] propanol | 48. [symbol] [b] | −0. [symbol] 70 |
| 1-He [symbol] ol | 48. [symbol] [ [symbol] ] | −0. [symbol] 0 |
| 1-Octanol | 4 [symbol] | −0. [symbol] 63 |
| Cyclop [symbol] l | 47.7 | −0. [symbol] 44 |
| 1-De [symbol] l | 4 [symbol] .6 [b] | −0. [symbol] |
| [symbol] -Dimethylphenol (2,6- [symbol] ) | 4 [symbol] . [symbol] | −0. [symbol] |
| 2-Buta [symbol] | 4 [symbol] . [symbol] | −0. [symbol] |
| 3-Methyl- [symbol] alcohol) | 4 [symbol] .0 | −0. [symbol] 1 |
| Cyclo [symbol] | 46. [symbol] | −0. [symbol] 8 |
| [symbol] -Dode [symbol] | 4 [symbol] . [symbol] [ [symbol] ] | −0. [symbol] 11 |
| [symbol] -Phen [symbol] nol | 4 [symbol] . [symbol] [ [symbol] ] | 0. [symbol] 11 |
| Acrylonitrile | 46.7 | −0. [symbol] 11 |
| 4-Methyl- [symbol] -dio [symbol] -one [symbol] | 46.6 | −0. [symbol] 08 |
| [symbol] | | |
| [symbol] -Pen [symbol] | [symbol] . [symbol] | −0.505 |
| Nitromethane | 4 [symbol] . [symbol] | −0.498 |
| Ace [symbol] ile | 46.0 | −0.489 |
| 3-Pentanol | 4 [symbol] . [symbol] | −0.479 |
| Dimethyl sulfoxide | 4 [symbol] .0 | −0.456 |
| Methyl acrylate | 44. [symbol] | −0.440 |
| A [symbol] | 44. [symbol] | −0.4 [symbol] 4 |
| Tetra- [symbol] -hexylammonium benzoate | 44. [symbol] | −0.4 [symbol] 4 |
| Tetrahydrothiophene [symbol] -dioxide | 44. [symbol] | −0. [symbol] |
| [symbol] ol [symbol] | | |
| 2-Methyl-2-propanol [symbol] butyl | 43.9 [g] | −0. [symbol] |
| alcohol [symbol] | | |
| Acetic anhydride | 43.9 [ [symbol] ] | −0. [symbol] |
| N,N-Dimethylformamide | 43.8 | −0.417 |
| N,N-Dimethyla [symbol] mide | 43.7 | −0.414 |
| Propionitrile | 43.7 | −0.414 |
| N [symbol] | 43.6 | −0.411 |
| Trimethyl phosphate | 43.6 | −0.411 |
| Buty [symbol] | 43. [symbol] | −0. [symbol] |
| Ben [symbol] cyanide | 42.9 | −0.388 |
| [symbol] -Methyla [symbol] | 42. [symbol] | −0. [symbol] 7 [symbol] |
| [symbol] -Methyl-2-pyr [symbol] ne | 42. [symbol] | −0.3 [symbol] |
| Acetone | 42. [symbol] | −0.3 [symbol] |

[symbol]

indicates data missing or illegible when filed

### REFERENCES

[1]J. F. Wright, AAV empty capsids: for better or for worse?Mol. Ther. 22 (2014) 1-2.
[2]C. Ling, Y. Wang, Y. Lu, L. Wang, G. R. Jayandharan, G. V. Aslanidi, B. Li, B. Cheng, W. Ma, T. Lentz, C. Ling, X. Xiao, R. J. Samulski, N. Muzyczka, A. Srivastava, The adeno-associated virus genome packaging puzzle, J. Mol. Genet. Med. 9 (2015) 175
[3]K. Gao, M. Li, L. Zhong, Q. Su, J. Li, S. Li, R. He, Y. Zhang, G. Hendricks, J. Wang, G. Gao, Empty virions in AAV8 vector preparations reduce transduction effi-ciency and may cause total viral particle dose-limiting side effects, Mol. Ther. Methods Clin. Dev. 1 (2014) 20139.
[4] Sihn C R, Handyside B, Liu S et al. Molecular analysis of AAV5-hFVIII-SQ vector-genome-processing kinetics in transduced mouse and nonhuman primate livers. Mol Ther Methods Clin Dev. 2021 Dec. 21; 24:142-153.
[5]J. F. Wright, AAV vector manufacturing process design and scalability—Bending the trajectory to address vector-associated immunotoxicities. Mol Ther. 2022 Jun. 1; 30(6):2119-2121.
[6] WO 2022/038164 A1.
[7]C. Reichardt, Empirical Parameters of Solvent Polarity as Linear Free-Energy Relationships. 1979 Jan Angewandte Chemie International Edition 18:98-110.
[8]S. Dukiie. F. Shoh, K:D. Nulre, R. Radeglia, Ukr. Khim. Zh (Russ. Ed.) 41, 1170 (1975); Chem. Abstr. 84, 430861 (1976).
[9] USP 621 Chromatography, 2022 December
[10] Lock M, Vandenberghe L H, Wilson J M. Scalable production method for AAV (US20160040137A1) 2016.
[11]P. Gagnon, B. Goricar, S. D. Prebil, H. Jug, M. Leskovec, A. Strancar, Separation of Empty and Full Adeno-Associated Virus Capsids from a Weak Anion Exchanger by Elution with an Ascending pH Gradient at Low Ionic Strength, 2021. https://biopro cessingjournal.com/afp/J200A-Gagnon.pdf (accessed May 25, 2022).
[12]P. Gagnon, M. Leskovec, S. D. Prebil, Rok Žigon, M. Štokelj, A. Raspor, S. Peljhan, A. Štrancar. J Chromatogr A. Removal of empty capsids from adeno-associated virus preparations by multimodal metal affinity chromatography 2021 Jul. 19; 1649:462210.
[13] Chen, R. F. Fluorescence quantum yield of tryptophan and tyrosine. Analyt. Lett. 1967, 1, 35-42.
[14] Ghisaidoobe, A. B. T.; Chung, S. A. Intrinsic tryptophan fluorescence in the detection and analysis of proteins: A focus on the Forster resonance energy transfer techniques. Intl. J. Mol. Sci. 2014, 15, 22518-22538.
[15] Singer, V.; Jones, L.; Yue, S. T.; Hauglund, R. P. Characterization of Picogreen reagent and development of a fluorescence-based solution assay for double-stranded DNA quantitation. Anal. Biochem. 1997, 249, 228-238.
[16] Yang, Yun; Geng, Xindu (2011). “Mixed-mode chromatography and its applications to biopolymers”. Journal of Chromatography A. 1218 (49): 8813-8825. doi:10.1016/j.chroma.2011.10.009. ISSN 0021-9673. PMID 22033107.
[17] Zhao, Guofeng; Dong, Xiao-Yan; Sun, Yan (2009). “Ligands for mixed-mode protein chromatography: Principles, characteristics and design”. Journal of Biotechnology. 144 (1): 3-11. doi:10.1016/j.jbiotec.2009.04.009. ISSN 0168-1656. PMID 19409941.
[18] McLaughlin, Larry W. (1989). “Mixed-mode chromatography of nucleic acids”. Chemical Reviews. 89 (2): 309-319. doi:10.1021/cr00092a003. ISSN 0009-2665.
[19] Peljhan S, Štokelj M, Prebil S D, Gagnon P, Strancar A. Multiple-parameter profiling of density gradient ultracentrifugation for characterization of empty and full capsid distribution in AAV preparations. Cell Gene Ther. Insights 2021; 7(2), 161-169.
[20] Hildegard Buning, Arun Srivastava Capsid Modifications for Targeting and Improving the Efficacy of AAV Vectors. Mol Ther Methods Clin Dev. 2019 12: 248-265.
[21] Pierce J. Ogden, Heric D. Kelsic, Sam Sinai, George M. Church Comprehensive AAV capsid fitness landscape reveals a viral gene and enables machine-guided design. Science 2019 Vol 366, Issue 6469 pp. 1139-1143.

1. A method for enriching full Adeno-Associated Virus (AAV) capsids from a mixture comprising full Adeno-Associated Virus (AAV) capsids, partially filled and/or empty AAV capsids by means of chromatography, comprising the steps of
contacting the mixture with a strong or weak anion exchanger material
eluting the loaded mixture by means of a neutral to an alkaline buffer comprising an organic modifier having a relative measure of solvent polarity from 0.4 to 0.8,
and collecting a fraction enriched with full AAV capsids.

2. The method of claim 1 wherein the organic modifier is selected from the group consisting of acetonitrile, 1-butanol, t-butanol, propylene carbonate, isopropanol, ethanol, methanol, propanol and mixtures thereof.

3. The method of claim 1 wherein the buffer comprises alkaline earth metal salts.

4. The method of claim 1 wherein the buffer has a pH value of from pH 7.0 to pH 10.50.

5. The method of claim 1 wherein the buffer comprises
isotonic substances selected from the group consisting of sucrose, sorbitol, mannitol, xylitol and mixtures thereof and/or
non-ionic surfactants, such as poloxamer 188.

6. The method of claim 1 wherein the strong or weak anion exchanger material is a
(i) strong or weak anion exchanger material with or without hydrogen bond properties and compounded with or without positively charged metal affinity ligands as a multi modal material,
(ii) a monolith anion exchanger
(iii) a monolith multimodal material,
(iv) a particulate anion exchanger,
(v) a particulate multimodal material,
(vi) an anion exchanger or multimodal material arranged in membranes, and/or
(vii) a particle packed anion exchanger or multimodal column and/or
(viii) a fibre chromatography anion exchanger or a multimodal fibre column.

7. The method of claim 1 wherein the AAV is selected from the serotypes AAV1, AAV2, AAV3, AAV4, AAV5, AAV6, AAV7, AAV8, AAV9, AAVrh.10, AAV11, AAV12, and of further serotypes such as hybrid serotypes.

8-14. (canceled)

15. A method for enriching full Adeno-Associated Virus (AAV) capsids from a mixture comprising full Adeno-Associated Virus (AAV) capsids, partially filled and/or empty AAV capsids by means of chromatography, comprising the steps of
contacting the mixture with a strong or weak anion exchanger material
eluting the loaded mixture by means of a neutral to an alkaline buffer comprising an organic modifier selected from the group consisting of methanol, 1,3-propanediol, 1,2-propanediol, N-methylformamide, diethylene glycol, triethylene glycol, 1,3-butanediol, 2-propyn-1-ol (propargyl alcohol), 2-methoxyethanol, 2-propen-1-ol (allyl alcohol), N-methylacetamide, ethanol, 2-aminoethanol, acetic acid, benzyl alcohol, 1-propanol, 1-butanol, 2-hydroxymethylfuran (furfuryl alcohol), 2-phenylethanol, 1-pentanol, 2-methyl-1-propanol (isobutyl alcohol), 1-hexanol, 2-propanol, 3-phenyl-1-propanol, 1-heptanol, 1-octanol, cyclopentanol, 1-decanol, 2,6-dimethylphenol (2,6-xylenol), 2-butanol, 3-methyl-1-butanol (isoamyl alcohol), cyclohexanol, 1-dodecanol, 1-phenylethanol, acrylonitrile, 4-methyl-1,3-dioxolan-2-one (propylene carbonate), 2-pentanol, nitromethane, acetonitrile, dimethyl sulfoxide, methyl acrylate, aniline, tetra-N-hexylammonium benzoate, tetrahydrothiophene 1,1-dioxide (sulfolane), 2-methyl-2-propanol (tert-butyl alcohol), acetic anhydride, N,N-dimethylformamide, N,N-dimethylacetamide, propionitrile and nitroethane,
and collecting a fraction enriched with full AAV capsids.

16. The method of claim 3 wherein the alkaline earth metal salts are magnesium acetate, magnesium formate, calcium acetate or calcium formate or mixtures thereof and/or their more kosmotropic alternatives.

17. The method of claim 7 wherein the hybrid serotypes are selected from a recombinant hybrid serotype, chimeras, surface modified AAVs, and synthetically derived AAV like particles.

18. The method of claim 17 wherein the recombinant hybrid serotype comprises AAV2/8.
